SEQUENCE LISTING (19 sequences)

SEQ ID NO: 1 - DNA, 1602 bp, Homo sapiens
  source 1..1602: mol_type = other DNA
atgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaatgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttga

SEQ ID NO: 2 - DNA, 1602 bp, Homo sapiens
  source 1..1602: mol_type = other DNA
atgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacacgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaacgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcacggactgttcaaaaaatcttga

SEQ ID NO: 3 - DNA, 1602 bp, Homo sapiens
  source 1..1602: mol_type = other DNA
atgtccatccaggtggagcacccagctggaggctacaagaagctgtttgaaactgtggaagaactgagcagccccctgacagcccatgtgacaggcagaatccctctgtggctgacaggcagcctgctgagatgtggcccaggcctgtttgaggtgggctctgagcctttctaccacctgtttgatggccaagccctgctccacaagtttgatttcaaggagggccatgtgacctaccacagaagattcatcagaacagatgcctatgtgagggccatgacagagaagaggatagttatcacagagtttggcacctgtgccttccctgacccctgcaagaacatcttcagcagattcttcagctacttcagaggagtggaagtgacagacaatgccctggtcaatgtgtaccctgtgggagaggactactatgcctgtactgagaccaacttcatcaccaagatcaaccctgaaaccctggaaaccatcaagcaggtggacctgtgcaactatgtgtcagtcaatggagccacagcccaccctcacattgagaatgatggcacagtttacaacataggcaactgctttggcaaaaacttcagcattgcctacaacattgtgaagatcccccctctgcaggctgacaaggaggaccccatcagcaagtctgagatagtggtgcagttcccatgctctgacagattcaagcccagctatgtgcacagctttggcctgaccccaaactacattgtgtttgtggaaacccctgtgaagatcaacctgttcaagttcctgagctcctggagcctgtggggagccaactacatggactgctttgaaagcaatgagaccatgggagtgtggctgcacattgctgacaagaaaagaaagaagtacctgaacaacaaatacagaaccagccctttcaacctgttccaccacatcaacacctatgaggacaatggcttcctgattgtggacctgtgctgctggaagggctttgagtttgtgtacaactacctgtacctggccaacctgagagaaaactgggaggaagtgaaaaaaaatgccagaaaggccccccagcctgaggtgaggagatatgtgctgcctctgaacatagacaaggctgacacaggcaagaacctggtgaccctccccaacaccacagccacagccatcctgtgctctgatgagaccatctggctggagcctgaagtgctgttctctggccccagacaggcctttgagttccctcaaatcaactaccagaaatactgtggcaaaccctacacctatgcctatggcctgggcctgaaccactttgtccctgacagactgtgcaagctgaatgtgaaaaccaaggagacctgggtctggcaggagcctgactcctacccttctgaacccatctttgtgagccaccctgatgccctggaggaggatgatggagtggtgctgagtgtggtggtcagccctggtgctggccagaagcctgcatacctgctaatcctgaatgccaaggacctgtctgaagttgccagggctgaggtggaaatcaacatccctgtgaccttccatggccttttcaagaagagctga

SEQ ID NO: 4 - DNA, 1602 bp, Homo sapiens
  source 1..1602: mol_type = other DNA
atgagcatccaggtggaacatcctgctggtggctacaagaaactgtttgagacagtggaagaactgagcagccctctgacagcccatgtgacaggcagaatccctctgtggctgacaggctccctgctgagatgtggccctggcctgtttgaagtgggctctgagcctttctaccacctgtttgatggacaggccctgctgcacaagtttgacttcaaagagggccatgtgacctaccacagaagattcatcaggacagatgcctatgtcagagccatgacagagaagaggattgtgatcactgagtttggcacctgtgcctttccagatccttgcaagaacatcttcagcagattcttcagctacttcagaggggttgaagtgacagacaatgccctggtcaatgtgtaccctgtgggagaagattactatgcctgcacagagacaaacttcatcaccaagatcaaccctgagacactggaaaccatcaagcaggttgacctgtgcaactatgtgtctgtgaatggggccacagctcaccctcacattgagaatgatggcacagtgtacaacattggcaactgctttggcaagaacttcagcattgcctacaacatagtgaagatcccacctctgcaggctgacaaagaggaccccatcagcaagtctgagattgtggtgcagttcccctgctctgacagattcaagcccagctatgtgcacagctttggcctgacacctaactacattgtgtttgtggaaacccctgtgaagatcaatctgttcaagttcctgagcagctggtccctgtggggagccaactacatggactgctttgagagcaatgagacaatgggagtgtggctgcacattgcagacaagaagagaaagaagtacctgaacaacaagtacaggacaagccccttcaacctgttccaccacatcaacacctatgaggacaatggcttcctgattgtggacctgtgctgctggaagggctttgagtttgtgtacaactacctgtacctggccaacctgagggaaaactgggaagaagtgaagaagaatgccagaaaggcccctcagcctgaagttagaagatatgtgctgcccctgaacattgacaaggctgacacaggcaagaacctggtcaccctgcctaacaccacagccacagccatcctgtgctctgatgagactatctggctggaacctgaggtgctgttctctggccccagacaggcctttgagttccctcagatcaactaccagaaatactgtggcaagccctacacctatgcctatggcctgggcctgaaccactttgtgccagacagactgtgcaagctgaatgtcaagaccaaagagacatgggtctggcaagagcctgacagctacccttctgagcccatctttgtgtctcaccctgatgctctggaagaggatgatggggttgtgctgtctgtggtggtgtcccctggtgctggacagaagcctgcctatctgctgatcctgaatgccaaggacctgtctgaggtggccagagctgaggtggaaatcaacatccctgtgaccttccatggcctgttcaagaagtcctga

SEQ ID NO: 5 - DNA, 1602 bp, Homo sapiens
  source 1..1602: mol_type = other DNA
atgagcatccaggtggaacatcctgctggtggctacaagaaactgtttgagacagtggaagaactgagcagccctctgacagcccatgtgacaggcagaatccctctgtggctgacaggctccctgctgagatgtggccctggcctgtttgaagtgggctctgagcctttctaccacctgtttgatggacaggccctgctgcacaagtttgacttcaaagagggccatgtgacctaccacagaagattcatcaggacagatgcctatgtcagagccatgacagagaagaggattgtgatcactgagtttggcacctgtgcctttccagatccttgcaagaacatcttcagcagattcttcagctacttcagaggggttgaagtgacagacaatgccctggtcaatgtgtaccctgtgggagaagattactatgcctgcacagagacaaacttcatcaccaagatcaaccctgagacactggaaaccatcaagcaggttgacctgtgcaactatgtgtctgtgaatggggccacagctcaccctcacattgagaatgatggcacagtgtacaacattggcaactgctttggcaagaacttcagcattgcctacaacatagtgaagatcccacctctgcaggctgacaaagaggaccccatcagcaagtctgagattgtggtgcagttcccctgctctgacagattcaagcccagctatgtgcacagctttggcctgacacctaactacattgtgtttgtggaaacccctgtgaagatcaatctgttcaagttcctgagcagctggtccctgtggggagccaactacatggactgctttgagagcaatgagacaatgggagtgtggctgcacattgcagacaagaagagaaagaagtacctgaacaacaagtaccggacaagccccttcaacctgttccaccacatcaacacctatgaggacaatggcttcctgattgtggacctgtgctgctggaagggctttgagttcgtgtacaactacctgtacctggccaacctgagggaaaactgggaagaagtgaagaagaatgccagaaaggcccctcagcctgaagttagaagatatgtgctgcccctgaacattgacaaggctgacacaggcaagaacctggtcaccctgcctaacaccacagccacagccatcctgtgctctgatgagactatctggctggaacctgaggtgctgttctctggccccagacaggccttcgagttccctcagatcaactaccagaaatactgcggcaagccctacacctatgcctatggcctgggcctgaaccacttcgtgccagacagactgtgcaagctgaatgtcaagaccaaagagacatgggtctggcaagagcctgacagctacccttctgagcccatctttgtgtctcaccctgatgctctggaagaggatgatggggttgtgctgtctgtggtggtgtcccctggtgctggacagaagcctgcctatctgctgatcctgaatgccaaggacctgtctgaggtggccagagctgaggtggaaatcaacatccctgtgaccttccatggcctgttcaagaagtcctga

SEQ ID NO: 6 - DNA, 1602 bp, Homo sapiens
  source 1..1602: mol_type = other DNA
atgtccatccaggtggagcacccagctggaggctacaagaagctgtttgagactgtggaagaactgagcagccccctgacagcccatgtgacaggcaggatccccctgtggctgacaggcagcctgctgagatgtggcccaggcctgtttgaggtgggctctgagcctttctaccacctgtttgatggccaagccctgctccacaagttcgatttcaaagagggccatgtgacctaccacagacggttcatcagaacagatgcctatgtgagagccatgacagagaagagaattgtgatcacagaatttggcacctgtgccttccctgacccctgcaagaacatcttcagcagattcttcagctacttcagaggagtggaagtgacagacaatgccctggtgaatgtgtaccctgtgggagaagactactatgcctgtactgagaccaacttcatcaccaagatcaaccctgaaaccctggaaaccatcaagcaggtggacctgtgcaactatgtgtcagtcaatggagccacagcccaccctcacatcgagaatgatggcacagtttacaacattggcaactgctttggcaaaaacttcagcatcgcctacaacattgtgaagatcccccctctgcaggctgacaaagaggaccccatcagcaagtctgagatagtggtgcagttcccatgctctgaccggttcaagcccagctatgtgcacagctttggcctgaccccaaactacattgtgtttgtggaaacccctgtcaaaatcaacctgttcaaattcctgagctcctggagcctgtggggagccaactacatggactgctttgaaagcaatgagaccatgggagtgtggctgcacattgctgacaagaaacggaagaagtacctgaacaacaagtaccggaccagccctttcaacctgttccaccacatcaacacctatgaggacaatggcttcctgatcgtggacctgtgctgctggaagggctttgagttcgtgtacaactacctgtacctggccaacctgagagaaaactgggaggaggtgaagaagaatgccagaaaggccccccagcctgaagtgaggagatatgtgctgcctctgaacatagacaaggctgacacaggcaagaacctggtgaccctccctaacaccacagccacagccatcctctgctctgatgagaccatctggctggaacctgaagtgctgttctctggccccagacaggcctttgagttcccacaaatcaactaccagaaatactgtggcaagccctacacctacgcctatggcctgggcctgaaccactttgtgcctgacagactgtgcaagctgaatgtgaagaccaaggagacctgggtctggcaggagcctgactcctacccttctgaacccatctttgtcagccaccctgatgccctggaggaggatgatggagtggtgctgagtgtggtggtgagccctggtgctggccagaagcctgcatacctgctgatcctgaatgccaaggacctgtctgaggttgccagagctgaggtggaaatcaacatccctgtcaccttccatggcttattcaagaaaagctga

SEQ ID NO: 7 - DNA, 1605 bp, Homo sapiens
  source 1..1605: mol_type = other DNA
atgagcatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaacgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttaataa

SEQ ID NO: 8 - DNA, 1602 bp, Homo sapiens
  source 1..1602: mol_type = other DNA
atgtctattcaagtcgagcacccagcggggggatataaaaagcttttcgaaacggtggaggagctgagctccccccttacggcgcatgttacggggcgcatacctctgtggctcacgggatcattgcttcgctgcggacccggattgttcgaggttggcagtgaaccattctaccatctcttcgatggtcaggcattgcttcataaatttgatttcaaagaaggacacgtcacatatcatcgcaggttcatccggacagatgcgtacgttcgcgccatgacagaaaagcgcattgtaataactgagtttgggacatgtgcatttcctgacccttgtaagaatatattcagccgctttttcagctattttagaggcgttgaggttactgacaatgcgctcgtgaacgtctatccagtaggtgaagattattacgcctgtactgagactaatttcatcactaaaattaatccagaaactttggagaccataaagcaggttgatctctgtaactatgtctcagtgaatggcgctacagcgcacccgcacatagaaaacgatggaacggtttacaatataggcaactgttttggtaagaattttagcattgcttacaacatagtcaagatacctcctctccaagccgataaagaggacccgatatccaaatcagagattgttgtccaattcccgtgcagtgatagattcaaaccgtcttacgtccacagttttggcctgacacccaattacattgtttttgttgaaacacctgttaagataaatctctttaaatttctgtcttcttggagtctgtggggggcaaattacatggattgtttcgagtctaacgagacgatgggagtctggcttcatatagcagataaaaagcgcaaaaagtatttgaataacaagtaccggacgagcccgttcaatttgttccaccatatcaacacttacgaggataacgggtttctgatcgtcgacctttgctgttggaaagggttcgagttcgtgtataactacctctacttggcgaaccttcgggaaaattgggaggaagttaagaagaacgcaagaaaggccccgcagccagaagtccgaaggtatgttctgccgttgaatatcgacaaagccgacactggaaagaacctcgttacgcttcccaataccacggctaccgcgatcttgtgcagtgacgaaacaatttggctggagcccgaggtgttgttttctggcccaaggcaagcctttgaattcccacagataaattatcaaaaatattgtggaaagccctacacctacgcttatggactcggtctcaaccattttgttccagatcgactttgcaagctgaatgtaaagaccaaagaaacctgggtttggcaagaacccgattcctaccccagtgaaccgatctttgtttcccatcccgacgccctcgaagaagacgacggagttgtcttgtccgttgtggtgagccccggtgcaggacagaagcccgcttatcttttgattcttaatgccaaagatttgtcagaagtagcgcgggccgaggtagagatcaacatacctgttactttccatgggttgttcaaaaagagttga

SEQ ID NO: 9 - DNA, 1605 bp, Homo sapiens
  source 1..1605: mol_type = other DNA
atgagcatccaggtggaacatcctgccggcggatacaagaaactgttcgagacagtggaagaactgagcagccctctgacagcccacgtgacaggcagaatccctctgtggctgaccggcagcctgctgagatgtggacctggcctgtttgaagtgggcagcgagcctttctaccacctgttcgatggacaggccctgctgcacaagttcgacttcaaagagggccacgtcacctaccaccggcggttcattagaaccgatgcctacgtgcgggccatgaccgagaagagaatcgtgatcaccgagttcggcacctgtgcctttccagatccttgcaagaacatcttcagccggttcttcagctacttcagaggcgtggaagtgaccgacaacgccctggtcaatgtgtaccccgtgggcgaagattactacgcctgcaccgagacaaacttcatcaccaagatcaaccccgagacactggaaaccatcaagcaggttgacctgtgcaactacgtgtccgtgaacggcgccacagctcaccctcacatcgagaatgatggcaccgtgtacaacatcggcaactgcttcggcaagaacttctctatcgcctacaatatcgtgaagatcccacctctgcaggccgacaaagaggaccccatcagcaagagcgagatcgtggtgcagttcccctgcagcgacagattcaagcccagctacgtgcacagcttcggcctgacacctaactacatcgtgttcgtggaaacccctgtgaagatcaatctgttcaagttcctgagcagctggtccctgtggggcgccaactacatggattgcttcgagagcaacgagacaatgggcgtgtggctgcacattgccgacaagaagcggaagaagtacctgaacaacaagtaccggacaagccccttcaacctgttccaccacatcaacacctacgaggacaacggcttcctgatcgtggacctgtgttgctggaagggcttcgagttcgtgtacaattacctgtacctggccaacctgcgcgagaactgggaagaagtgaagaagaacgcccggaaggcccctcagcctgaagtgcgaagatatgtgctgcccctgaacatcgacaaggccgacaccggcaagaatctggtcaccctgcctaataccaccgccaccgccatcctgtgtagcgacgaaaccatctggctggaacccgaggtgctgttctctggacctagacaggccttcgagtttccccagatcaactaccagaagtactgcggcaagccctacacctacgcctatggcctgggcctgaatcacttcgtgcccgacagactgtgcaagctgaacgtcaagaccaaagagacatgggtctggcaagagcccgacagctaccctagcgagcccatctttgtgtctcaccccgacgctctggaagaggacgatggcgttgtgctgagcgtggtggtttctcctggcgccggacagaaacctgcctacctgctgatcctgaacgccaaggacctgagcgaagtggccagagccgaggtggaaatcaacatccccgtgaccttccacggcctgttcaagaagtcctaataa

SEQ ID NO: 10 - DNA, 1602 bp, Homo sapiens
  source 1..1602: mol_type = other DNA
atgagcatccaggtggagcaccccgccggcggctacaagaagctgttcgagaccgtggaggagctgagcagccccctgaccgcccacgtgaccggccgcatccccctgtggctgaccggcagcctgctgcgctgcggccccggcctgttcgaggtgggcagcgagcccttctaccacctgttcgacggccaggccctgctgcacaagttcgacttcaaggagggccacgtgacctaccaccgccgcttcatccgcaccgacgcctacgtgcgcgccatgaccgagaagcgcatcgtgatcaccgagttcggcacctgcgccttccccgacccctgcaagaacatcttcagccgcttcttcagctacttccgcggcgtggaggtgaccgacaacgccctggtgaacgtgtaccccgtgggcgaggactactacgcctgcaccgagaccaacttcatcaccaagatcaaccccgagaccctggagaccatcaagcaggtggacctgtgcaactacgtgagcgtgaacggcgccaccgcccacccccacatcgagaacgacggcaccgtgtacaacatcggcaactgcttcggcaagaacttcagcatcgcctacaacatcgtgaagatcccccccctgcaggccgacaaggaggaccccatcagcaagagcgagatcgtggtgcagttcccctgcagcgaccgcttcaagcccagctacgtgcacagcttcggcctgacccccaactacatcgtgttcgtggagacccccgtgaagatcaacctgttcaagttcctgagcagctggagcctgtggggcgccaactacatggactgcttcgagagcaacgagaccatgggcgtgtggctgcacatcgccgacaagaagcgcaagaagtacctgaacaacaagtaccgcaccagccccttcaacctgttccaccacatcaacacctacgaggacaacggcttcctgatcgtggacctgtgctgctggaagggcttcgagttcgtgtacaactacctgtacctggccaacctgcgcgagaactgggaggaggtgaagaagaacgcccgcaaggccccccagcccgaggtgcgccgctacgtgctgcccctgaacatcgacaaggccgacaccggcaagaacctggtgaccctgcccaacaccaccgccaccgccatcctgtgcagcgacgagaccatctggctggagcccgaggtgctgttcagcggcccccgccaggccttcgagttcccccagatcaactaccagaagtactgcggcaagccctacacctacgcctacggcctgggcctgaaccacttcgtgcccgaccgcctgtgcaagctgaacgtgaagaccaaggagacctgggtgtggcaggagcccgacagctaccccagcgagcccatcttcgtgagccaccccgacgccctggaggaggacgacggcgtggtgctgagcgtggtggtgagccccggcgccggccagaagcccgcctacctgctgatcctgaacgccaaggacctgagcgaggtggcccgcgccgaggtggagatcaacatccccgtgaccttccacggcctgttcaagaagagctaa

SEQ ID NO: 11 - protein, 533 aa, Homo sapiens
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 12 - DNA, 1604 bp, Homo sapiens
  source 1..1604: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgattgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccaattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatcgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 13 - DNA, 479 bp, Homo sapiens
  source 1..479: mol_type = other DNA
gggtggcatccctgtgacccctccccagtgcctctcctggccctggaagttgccactccagtgcccaccagccttgtcctaataaaattaagttgcaatccattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 14 - DNA, 225 bp, Homo sapiens
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 15 - DNA, 135 bp, Homo sapiens
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 16 - DNA, 589 bp, Homo sapiens
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 17 - protein, 735 aa, Homo sapiens [sequence omitted]

SEQ ID NO: 18 - protein, 598 aa, Homo sapiens
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 19 - protein, 533 aa, Homo sapiens
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

(19) United States
(12) Patent Application Publication
(10) Pub. No.: US 2023/0321280 A1
(43) Pub. Date: Oct. 12, 2023

(54) COMPOSITIONS AND METHODS FOR THE TREATMENT OF OCULAR DISEASES

(71) Applicant: Frontera Therapeutics, Inc., Bedford, MA (US)

(72) Inventors: Wei ZHAO, Boston, MA (US); Zhongdong SHI, Bedford, MA (US); Sebastian AGUIRRE KOZLOUSKI, Bedford, MA (US)

(21) Appl. No.: 18/157,577

(22) Filed: Jan. 20, 2023

Related U.S. Application Data
(63) Continuation of application No. PCT/CN2021/107284, filed on Jul. 20, 2021.

(30) Foreign Application Priority Data
Jul. 21, 2020 (CN) 202010704946.1
Apr. 8, 2021 (CN) 202110378979.6

(51) Int. Cl. A61K 48/00; A61K 38/52; A61P 27/02; C12N 9/18; C12N 15/86

(52) U.S. Cl. CPC A61K 48/0058; A61K 38/52; A61P 27/02; C12N 9/18; C12Y 301/01064; C12N 15/86; C12N 2750/14143; C12N 2750/14152; C12N 2750/14171

(57) ABSTRACT

The present disclosure relates to a pharmaceutical composition for the treatment of Leber congenital amaurosis, and a method for treating Leber congenital amaurosis using the pharmaceutical composition.

### RELATED APPLICATIONS

This application is a continuation of International Application No. PCT/CN2021/107284, filed Jul. 20, 2021, which claims the benefit of priority of Chinese patent application 202010704946.1 filed Jul. 21, 2020, and Chinese patent application 202110378979.6 filed Apr. 8, 2021, each of which is incorporated herein by reference in its entirety.

### SEQUENCE LISTING

The instant application contains a Sequence Listing which has been submitted electronically in XML format and is hereby incorporated by reference in its entirety. Said copy, created on Jan. 20, 2023, is named 57837-706301_SL.xml and is 38,733 bytes in size.

### BACKGROUND

Leber congenital amaurosis (LCA) is a rare hereditary ocular disease, which manifests as severe visual impairment at birth or early in life, and complete loss of vision typically occurring within the first 20 years. The manifestations of LCA are different depending on the affected parts and the associated genetic mutations.

Retinal pigment epithelium-specific 65 kDa protein (RPE65), also referred to as retinoid isomerohydrolase, is 65 kDa in size and encoded in humans by the RPE65 gene. RPE65 is an enzyme in the visual cycle of vertebrates, which is expressed in retinal pigment epithelium (RPE), and is also present in rod cells and cone cells. The defect of RPE65 may result in LCA, which accounts for about 6% to 16% of LCA cases.

### SUMMARY OF THE INVENTION

At present, there is a need in the art to develop drugs and methods that can effectively treat LCA. The present disclosure provides for the composition, pharmaceutical composition and method that can effectively treat inheritated eye disease such as LCA.

In one aspect, the present disclosure provides a recombinant adeno-associated virus (rAAV) particle, comprising an expression cassette polynucleotide sequence that comprises a coding sequence of RPE65 polypeptide, wherein the coding sequence is codon-optimized and contains an altered number of CpG dinucleotides as compared to a wild type RPE65 nucleotide sequence (SEQ ID NO: 1).

In some embodiments, the coding sequence comprises a reduced number of CpG dinucleotides as compared to the wild type RPE65 nucleotide sequence. In some embodiments, the coding sequence comprises about 50% of CpG dinucleotides as compared to the wild type RPE65 nucleotide sequence. In some embodiments, the coding sequence comprises no more than 20 CpG dinucleotides. In some embodiments, the coding sequence comprises no more than 10 CpG dinucleotides. In some embodiments, the coding sequence does not comprise CpG dinucleotides.

In some embodiments, the coding sequence comprises an increased number of CpG dinucleotides as compared to the wild type RPE65 nucleotide sequence. In some embodiments, the coding sequence comprises about 600% of CpG dinucleotides as compared to the wild type RPE65 nucleotide sequence. In some embodiments, the coding sequence comprises about 100 to 200 CpG dinucleotides. In some embodiments, the coding sequence is selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. In some embodiments, the coding sequence has at least 80% identity to one or more of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. In some embodiments, the coding sequence has at least 90% identity to one or more of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. In some embodiments, the coding sequence has at least 95% identity to one or more of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. In some embodiments, the coding sequence has at least 98% identity to one or more of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. In some embodiments, the coding sequence has at least 99% identity to one or more of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

In some embodiments, the rAAV particle further comprises an AAV capsid protein. In some embodiments, the AAV protein is from serotype AAV2 or variants thereof, serotype AAV5 or variants thereof, or serotype AAV8 or variants thereof.

In some embodiments, the expression cassette polynucleotide sequence further comprises a promoter, and the promoter is operably linked to the coding sequence. In some embodiments, the promoter is CMV, CAG, MNDU3, PGK, EF1a, Ubc promoter or ocular tissue specific promoter. In some embodiments, the ocular tissue specific promoter is selected from the RPE 65 gene promoter, human retinal binding protein (CRALBP) gene promoter, murine 11-cis-retinol dehydrogenase (RDH) gene promoter, rhodopsin promoter, rhodopsin kinase promoter, tissue inhibitor of metalloproteinase 3 (Timp3) promoter, photoreceptor retinol binding protein promoter and vitelliform macular dystrophy 2 promoter, or interphotoreceptor retinoid-binding protein (IRBP) promoters.

In some embodiments, the expression cassette sequence further comprises a WPRE sequence at the 3′ end. In some embodiments, the coding sequence further comprises a poly(A) sequence at the 3′ end. In some embodiments, the poly(A) sequence is one of SV40 late poly(A) (SV40pA), human growth hormone poly(A) (hGHpA), and bovine growth hormone poly(A) (bGHpA). In some embodiments, the polynucleotide further comprises a stuffer sequence. In some embodiments, the polynucleotide further comprises an inverted terminal repeat (ITR) sequence. In some embodiments, the inverted terminal repeat (ITR) sequence is a variant inverted terminal repeat (ITR) sequence.

In some embodiments, the polynucleotide comprises no more than 300 CpG dinucleotides. In some embodiments, the polynucleotide comprises no more than 250 CpG dinucleotides. In some embodiments, the polynucleotide comprises about 200 to 500 CpG dinucleotides.

In some embodiments, the polynucleotide further comprises sequences encoding other therapeutic proteins. In some embodiments, the other therapeutic proteins are selected from the group consisting of ABCA4, RDH12, RDH8, RBP3, RBP1, LRAT, RLBP1, RDH10 or RDH11. In some embodiments, the coding sequence is connected with the sequences encoding the other therapeutic proteins by a sequence encoding a linker. In some embodiments, the linker is a cleavable linker. In some embodiments, the linker comprises a sequence of a 2A peptide.

In another aspect, the present disclosure provides a composition comprising: (i) a first polynucleotide encoding an adeno-associated virus (AAV) protein, and (ii) a second polynucleotide comprising a sequence encoding a RPE65 polypeptide, wherein the sequence is codon-optimized and contains an altered number of CpG dinucleotides as compared to a wild type RPE65 nucleotide sequence.

In some embodiments, the RPE65 coding sequence comprises a reduced number of CpG dinucleotides as compared to the wild type RPE65 nucleotide sequence. In some embodiments, the sequence comprises about 50% of CpG dinucleotides as compared to the wild type RPE65 nucleotide sequence. In some embodiments, the sequence comprises no more than 20 CpG dinucleotides. In some embodiments, the sequence comprises no more than 10 CpG dinucleotides. In some embodiments, the sequence does not comprise CpG dinucleotides.

In some embodiments, the AAV capsid protein is from serotype AAV2 or variants thereof, serotype AAV5 or variants thereof, or serotype AAV8 or variants thereof 7 In some embodiments, the first polynucleotide is codon-optimized.

In some embodiments, the second polynucleotide comprises a promoter, and the promoter is operably linked to the sequence. In some embodiments, the promoter is CMV, CAG, MNDU3, PGK, EF1a, Ubc promoter or ocular tissue specific promoter. In some embodiments, the ocular tissue-specific promoter is selected from the RPE 65 gene promoter, human retinal binding protein (CRALBP) gene promoter, murine 11-cis-retinol dehydrogenase (RDH) gene promoter, rhodopsin promoter, rhodopsin kinase promoter, tissue inhibitor of metalloproteinase 3 (Timp3) promoter, photoreceptor retinol binding protein promoter and vitelliform macular dystrophy 2 promoter, or interphotoreceptor retinoid-binding protein (IRBP) promoters.

In some embodiments, the second polynucleotide comprises no more than 300 CpG dinucleotides. In some embodiments, the second polynucleotide comprises no more than 250 CpG dinucleotides. In some embodiments, the second polynucleotide comprises about 200 to 500 CpG dinucleotides.

In some embodiments, the sequence further comprises a WPRE sequence at the 3′ end. In some embodiments, the sequence further comprises a poly(A) sequence at the 3′ end. In some embodiments, the poly(A) sequence is one of SV40pA, hGHpA and bGHpA.

In some embodiments, the second polynucleotide further comprises a stuffer sequence. In some embodiments, the second polynucleotide further comprises an inverted terminal repeat (ITR) sequence. In some embodiments, the inverted terminal repeat (ITR) sequence is a variant inverted terminal repeat (ITR) sequence. In some embodiments, the second polynucleotide further comprises sequences encoding other therapeutic proteins. In some embodiments, the other therapeutic proteins are selected from the group consisting of ABCA4, RDH12, RDH8, RBP3, RBP1, LRAT, RLBP1, RDH10 or RDH11. In some embodiments, the sequence is connected with the sequences encoding the other therapeutic proteins by a sequence encoding a linker. The linker is a cleavable linker. In some embodiments, the linker comprises a sequence of a 2A peptide.

In another aspect, the present disclosure provides a method for preparing the recombinant adeno-associated virus (rAAV) particle, comprising introducing the herein described expression cassette polynucleotide sequence in a host cell. In another aspect, the present disclosure provides a recombinant adeno-associated virus (rAAV) particle, which is prepared by a method that comprises introducing the herein described expression cassette polynucleotide sequence in a host cell. In some embodiments, the method comprises expressing the herein described expression cassette polynucleotide sequence in the host cell. In some embodiments, the host cell is a human cell, animal cell, or insect cell. In some embodiments, the host cell is the Sf9 cell. In some embodiments, the host cell is the HEK293 cell or a derivative thereof. In some embodiments, the host cell is the HEK293T cell. In some embodiments, the method comprises generating bacmid DNA and/or baculovirus. In some embodiments, the method comprises generating RPE65 expression sequence bacmid DNA. In some embodiments, the method comprises generating rAAV cap expression sequence bacmid DNA. In some embodiments, the method comprises transfecting a host cell with the bacmid DNA to produce baculoviruses. In some embodiments, the method comprises transfecting a host cell with the RPE65 expression sequence bacmid DNA to produce baculoviruses. In some embodiments, the method comprises transfecting a host cell with the rAAV cap expression sequence bacmid DNA to produce baculoviruses. In some embodiments, the method further comprises mixing the two baculoviruses to infect a host cell (such as the Sf9 cell) to obtain packaged rAAV/RPE65-optimized virus particles of the present disclosure.

In another aspect, the present disclosure provides a pharmaceutical composition for treating Leber congenital amaurosis (LCA) in a subject in need thereof, which comprises the rAAV particle of the present disclosure and a pharmaceutically acceptable carrier.

In another aspect, the present disclosure provides a kit comprising the pharmaceutical composition of the present disclosure for treating LCA and instructions.

In another aspect, the present disclosure provides a pharmaceutical composition for treating Leber congenital amaurosis (LCA) in a subject in need thereof, which comprises administering a therapeutically effective amount of the rAAV particle or pharmaceutical composition of the present disclosure to the subject. In some embodiments, the therapeutically effective amount of the rAAV particle or pharmaceutical composition is administered by intravitreal injection, subretinal injection, or suprachoroidal injection. In some embodiments, the therapeutically effective amount is 1×10⁹−1×10¹³ of the rAAV particle. In some embodiments, the therapeutically effective amount is 1×10⁹−1×10¹³ of vector genomes (vg) for each eye. In another aspect, the present disclosure provides the use of an rAAV particle as described herein in the preparation of a medicament for treating an eye disease associated with a mutation of RPE65. In another aspect, the present disclosure provides the use of an rAAV particle as described herein in the preparation of a medicament for treating an inherited retinal disease (IRD) in a subject. In some embodiments, the IRD is associated with a mutation of RPE65. In some embodiments, the IRD is due to mutations in both copies of RPE65 gene in the subject. In some embodiments, the IRD is due to one or more mutations in one copy of RPE65 gene in the subject. In some embodiments, the IRD is LCA.

### BRIEF DESCRIPTION OF THE DRAWINGS

FIG. 1 is a schematic diagram of a part of polynucleotide expression cassette structures containing optimized RPE65 coding sequence.

### DETAILED DESCRIPTION

While various embodiments of the disclosure have been shown and described herein, it will be apparent to those skilled in the art that these embodiments are provided by way of example only. Many variations, changes and substitutions will occur to those skilled in the art without departing from the disclosure. It should be understood that various alternatives to the embodiments of the disclosure described herein may be employed.

Unless otherwise indicated, the practice of some embodiments disclosed herein employs conventional techniques of immunology, biochemistry, chemistry, molecular biology, microbiology, cell biology, genomics, and recombinant DNA. See, for example, Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PC 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

### Definitions

As used in the specification and claims, the singular forms “a”, “an” and “the” include plural reference unless the context clearly dictates otherwise. For example, the term “an rAAV particle” includes one or more rAAV particles.

The term “about” or “approximately” means within an acceptable error range of a specific value as determined by a person of ordinary skill in the art, which will depend in part on how the value is measured or determined, i.e., the limitations of the measurement system. For example, according to the practice in the art, “about” may mean within 1 or more than 1 standard deviation. Alternatively, “about” may mean a range of up to 20%, up to 10%, up to 5%, or up to 1% of a given value. Alternatively, particularly with respect to biological systems or processes, the term may mean within an order of magnitude, preferably within 5-fold, and more preferably within 2-fold, of the value. Where a specific value is described in the application and claims, it should be assumed that the term “about” means within an acceptable error range of the specific value unless otherwise stated.

As used herein, the terms “polypeptide”, “peptide” and “protein” are used interchangeably herein to refer to amino acid polymers of any length. The polymer can be linear, cyclic or branched, which can contain modified amino acids, and can be interrupted by non-amino acids. The terms also include amino acid polymers that have been modified, such as by sulfation, glycosylation, acetylation, acetylation, phosphorylation, iodination, methylation, oxidation, proteolysis, phosphorylation, isoprenylation, racemization, selenization, transfer RNA-mediated addition of amino acids to proteins (e.g., arginylation), ubiquitination, or any other operations, such as conjugation with labelling components. As used herein, the term “amino acid” refers to natural and/or unnatural or synthetic amino acids, including glycine and D or L optical isomers, as well as amino acid analogs and peptidomimetics. A polypeptide or amino acid sequence “derived” from a given protein refers to the origin of the polypeptide. Preferably, the polypeptide has an amino acid sequence that is substantially the same as the amino acid sequence of the polypeptide encoded in the sequence, or a portion thereof, wherein the portion consists of at least 10-20 amino acids or at least 20-30 amino acids or at least 30-50 amino acids, or can be identified immunologically with the polypeptide encoded in the sequence. The term also includes polypeptides expressed from a given nucleic acid sequence. As used herein, the term “domain” refers to a portion of a protein that is physically or functionally distinguished from other portions of the protein or peptide. Physically defined domains include amino acid sequences that are extremely hydrophobic or hydrophilic, such as those that are membrane-bound or cytoplasmic-bound. Domains can also be defined, for example, by internal homology caused by gene replication. Functionally defined domains have different biological functions. For example, an antigen-binding domain refers to an antigen-binding unit or a portion of an antibody that binds to the antigen. Functionally defined domains need not be encoded by continuous amino acid sequences, and functionally defined domains may contain one or more physically defined domains.

As used herein, the term “amino acid” refers to natural and/or unnatural or synthetic amino acids, including but not limited to D or L optical isomers, as well as amino acid analogs and peptidomimetics. Standard one-letter or three-letter codes are used to designate amino acids. Amino acids are typically denoted herein by one-letter and three-letter abbreviations well known in the art. For example, alanine may be represented by A or Ala.

As used herein, in the case of polypeptides, a “sequence” is the sequence of amino acids in the polypeptide in the direction from the amino terminus to the carboxy terminus, wherein the residues adjacent to each other in the sequence are contiguous in the primary structure of the polypeptide. The sequence may also be a linear sequence of a portion of a polypeptide known to contain additional residues in one or two directions.

As used herein, “identity”, “homology” or “sequence identity” refers to sequence similarity or interchangeability between two or more polynucleotide sequences or between two or more polypeptide sequences. When sequence identity, similarity or homology between two different amino acid sequences is determined using programs such as Emboss Needle or BestFit, a default setting may be used, or an appropriate scoring matrix, such as blosum45 or blosum80, may be selected to optimize the score of identity, similarity, or homology. Preferably, homologous polynucleotides are those that hybridize under stringent conditions as defined herein and have a sequence identity of at least 70%, preferably at least 80%, more preferably at least 90%, more preferably 95%, more preferably 97%, more preferably 98% and even more preferably 99% compared to these sequences. When sequences of comparable lengths are optimally aligned, the homologous polypeptides preferably have at least 80%, or at least 90%, or at least 95%, or at least 97%, or at least 98% sequence identity, or have at least 99% sequence identity.

As used herein, the “percentage sequence identity (%)” is defined as the percentage of amino acid residues in the query sequence that are identical to the amino acid residues of a second reference polypeptide sequence or a portion thereof after aligning the sequences and introducing gaps if necessary to obtain the maximum percentage sequence identity, and without taking any conservative substitutions as part of sequence identity. The alignment aimed at determining the percentage of amino acid sequence identity can be achieved in various ways within the skill of the art, for example, by using publicly available computer software, such as BLAST, BLAST-2, ALIGN, NEEDLE or Megalign (DNASTAR) software. Those skilled in the art can determine the appropriate parameters for measuring the alignment, including any algorithm required to obtain the maximum alignment over the full length of the sequences being compared. The percentage identity may be measured over the length of the entire defined polypeptide sequence, or may be measured over a shorter length, for example, the length of a fragment taken from a larger defined polypeptide sequence, such as fragments of at least 5, at least 10, at least 15, at least 20, at least 50, at least 100, or at least 200 consecutive residues. These lengths are only exemplary, and it should be understood that any fragment length supported by the sequences shown in the tables, drawings, or sequence listing herein can be used to describe the length over which the percentage identity can be measured.

The proteins described herein may have one or more modifications relative to a reference sequence. The modifications may be deletion, insertion or addition, or substitution or replacement of amino acid residues. “Deletion” refers to a change in amino acid sequence due to the lack of one or more amino acid residues. “Insertion” or “addition” refers to a change in amino acid sequence due to the addition of one or more amino acid residues compared to a reference sequence. “Substitution” or “replacement” refers to the substitution of one or more amino acids with different amino acids. Herein, the mutation of the antigen-binding unit relative to a reference sequence can be determined by comparing the antigen-binding unit with the reference sequence. The optimal alignment of sequences for comparison can be performed according to any known method in the art.

As used herein, the term “isolated” refers to being isolated from cellular and other components with which polynucleotides, peptides, polypeptides, proteins, antibodies or fragments thereof are associated under normal circumstances in nature. Those skilled in the art know that non-naturally occurring polynucleotides, peptides, polypeptides, proteins, antibodies or fragments thereof need not be “isolated” to distinguish from their naturally occurring counterparts. In addition, “concentrated”, “isolated” or “diluted” polynucleotides, peptides, polypeptides, proteins, antibodies or fragments thereof are distinguishable from their naturally occurring counterparts because the concentration or number of molecules per unit volume is greater than (“concentrated”) or less than (“isolated”) their naturally occurring counterparts. Enrichment may be measured based on absolute amounts, such as the weight of solution per unit volume, or it can be measured relative to a second, potentially interfering species present in the source mixture.

The terms “polynucleotide”, “nucleic acid”, “nucleotide” and “oligonucleotide” are used interchangeably. They refer to polymeric forms of nucleotides (whether deoxyribonucleotides or ribonucleotides) or their analogs of any length. Polynucleotides may have any three-dimensional structure, and may perform any known or unknown function. The following are non-limiting examples of polynucleotides: coding or non-coding regions of genes or gene fragments, loci determined from linkage analysis, exons, introns, messenger RNAs (mRNAs), transfer RNAs, ribosomal RNAs, ribozymes, cDNAs, recombinant polynucleotides, branched polynucleotides, plasmids, vectors, isolated DNAs of any sequence, isolated RNAs of any sequence, nucleic acid probes, primers, oligonucleotides or synthetic DNAs. Polynucleotides may contain modified nucleotides, such as methylated nucleotides and nucleotide analogs. Modifications to the nucleotide structure, if present, can be imparted before or after assembly of the polymer. The sequence of nucleotides may be interrupted by non-nucleotide components. The polynucleotide may be further modified after polymerization, for example by conjugation with a labeling component.

When applied to polynucleotides, “recombinant” means that the polynucleotide is the product of various combinations of cloning, restriction digestion and/or ligation steps, and other procedures that produce constructs different from polynucleotides found in nature.

The terms “gene” or “gene fragment” are used interchangeably herein. They refer to polynucleotides comprising at least one open reading frame that can encode a specific protein after transcription and translation. The gene or gene fragment may be genomic, cDNA or synthetic, as long as the polynucleotide comprises at least one open reading frame, which may cover the entire coding region or a segment thereof.

The term “operably linked” or “effectively linked” refers to juxtaposition, where the components so described are in a relation that allows them to function in their intended manner. For example, if a promoter sequence promotes the transcription of a coding sequence, the promoter sequence is operably linked to the coding sequence.

As used herein, “expression” refers to the process by which polynucleotides are transcribed into mRNAs, and/or the process by which transcribed mRNAs (also referred to as “transcripts”) are subsequently translated into peptides, polypeptides or proteins. The transcripts and the encoded polypeptides are collectively referred to as gene products. If a polynucleotide is derived from genomic DNA, expression may include splicing of mRNA in eukaryotic cells.

As used herein, the term “vector” refers to a tool for nucleic acid delivery, into which polynucleotides can be inserted. When a vector can express the protein encoded by the inserted polynucleotide, the vector is called an expression vector. A vector can be introduced into a host cell through transformation, transduction or transfection, so that the genetic material elements it carries can be expressed in the host cell. Vectors are well known to those skilled in the art, including but not limited to: plasmids; phagemids; artificial chromosomes, such as yeast artificial chromosomes (YAC), bacterial artificial chromosomes (BAC) or P1-derived artificial chromosomes (PAC); bacteriophages such as lambda bacteriophage or M13 bacteriophage and animal viruses and the like. Animal viruses that can be used as vectors include but are not limited to reverse transcriptase viruses (including lentiviruses), adenoviruses, adeno-associated viruses, herpes viruses (e.g., herpes simplex virus), poxviruses, baculoviruses, papilloma viruses, and papovaviruses (e.g., SV40). A vector may contain a variety of elements that control expression, including, but not limited to, promoter sequences, transcription initiation sequences, enhancer sequences, selection elements, and reporter genes. In addition, a vector may also contain origin of replication sites.

As used herein, the term “host cell” refers to a cell that can be used to introduce a vector, which includes, but is not limited to, prokaryotic cells such as Escherichia coli or Bacillus subtilis, fungal cells such as yeast cells or Aspergillus, insect cells such as S2 Drosophila cells or Sf9, or animal and human cells such as fibroblasts, CHO cells, COS cells, NSO cells, HeLa cells, BHK cells, HEK293 cells, or derivatives thereof.

As used herein, “effective amount refers to at least the minimum amount required to achieve a measurable improvement or prevention of a particular condition. The effective amount herein may vary with the patient's disease state, age, gender, weight and other factors. An effective amount is also an amount in which the therapeutic benefit exceeds any toxic or adverse effects in treatment. In the treatment of cancer or tumors, the effective amount of the drug can have the following effects: reducing the number of cancer cells, reducing tumor size, inhibiting cancer cell infiltration into peripheral organs, inhibiting tumor metastasis, inhibiting tumor growth to a certain extent, and/or alleviating one or more symptoms related to diseases to a certain extent. The effective amount can be administered in one or more dose.

As used herein, the terms “recipient”, “individual”, “subject”, “host”, and “patient” are used interchangeably herein, and refer to any mammalian subject, particularly humans, for whom diagnosis, treatment or therapy is desired.

As used herein, the terms “therapy” and “treatment” refer to obtaining a desired pharmacological and/or physiological effect. The effect may be prophylactic in terms of completely or partially preventing a disease or its symptoms, and/or may be therapeutic in terms of partially or completely stabilizing or curing the disease and/or adverse reactions attributed to the disease. As used herein, “treatment” encompasses any treatment of a disease in a mammal, such as mice, rats, rabbits, pigs, primates, including humans and other apes, especially humans, and the term includes: (a) preventing a disease or symptom from occurring in subjects who may be susceptible to the disease or symptom but are not yet diagnosed; (b) inhibiting a disease symptom; (c) preventing the development of the disease; (d) alleviating a disease symptom; (e) causing regression of a disease or symptom; or any combination thereof The term “kit” as used herein refers to a combination packaged for use together or commercially available. For example, the kit of the present disclosure may include the composition of the present disclosure, and instructions for using the composition or the kit. The term “instructions” refers to the explanatory inserts usually contained in commercial packages of therapeutic products, which contain information about indications, use, dosage, administration, combination therapies, contraindications and/or warnings about the use of such therapeutic products.

“Codon optimization” refers to changing the codons that make up a nucleic acid sequence so that the codons are most suitable for expression in a specific system (e.g., a specific species or a group of species). For example, a nucleic acid sequence is optimized for more efficient expression in mammalian cells. Due to the existence of synonymous codons, codon optimization does not change the amino acid sequence of the encoded protein. A variety of codon optimization methods are known in the art, such as those disclosed in U.S. Pat. Nos. 5,786,464 and 6,114,148. “Synonymous codons” refer to codons that encode the same amino acid.

### Leber Congenital Amaurosis and Other Disease

In one aspect, provided herein are compositions and methods for treating a disease or condition in a subject. The disease or condition can be an inherited retinal disease (IRD). In some embodiments, the IRD is caused by mutations of the RPE65 gene. In some embodiments, the IRD is caused by mutations of both copies of the RPE65 gene in the subject. In some embodiments, enough viable cells remain in the retina of the subject. In some embodiments, the disease or condition is Leber congenital amaurosis (LCA).

Leber congenital amaurosis (LCA) is a rare hereditary ocular disease, accounting for about 6% of all hereditary retinal diseases, and is the most serious form of hereditary retinopathy. LCA is the most common cause of congenital blindness in children, which usually manifests as severe visual impairment at birth or early in life, and complete loss of vision occurring within the first 20 years. During the development of LCA, the symptoms of the patient's disease include retinal dysfunction, eye movement (nystagmus), visual impairment, pupil unresponsiveness, and eventually blindness.

LCA is usually an autosomal recessive genetic disease. To date, 18 genes related to LCA have been identified, and mutations in these genes are usually the cause of LCA. According to these 18 genes, the Online Mendelian Inheritance In Man (OMIM) further divides LCA into 18 different types. The different types of LCA and the genetic information associated therewith are shown in Table 1 below.

TABLE 1
Classification of LCA and related genes

| Type | Gene | Locus |
|---|---|---|
| LCA1 | GUCY2D | 17p13.1 |
| LCA2 | RPE65 | 1p31.3-p31.2 |
| LCA3 | SPATA7 | 14q31.3 |
| LCA4 | AIPL1 | 17p13.2 |
| LCA5 | LCA5 | 6q14.1 |
| LCA6 | RPGRIP1 | 14q11.2 |
| LCA7 | CRX | 19q13.3 |
| LCA8 | CRB1 | 1q31-q32.1 |
| LCA9 | NMNAT1 | 1p36.22 |
| LCA10 | CEP290 | 12q21.32 |
| LCA11 | IMPDH1 | 7q32.1 |
| LCA12 | RD3 | 7q32.1 |
| LCA13 | RDH12 | 1q32.3 |
| LCA14 | LRAT | 14q24.1 |
| LCA15 | TULP1 | 4q31 |
| LCA16 | KCNJ13 | 2q37 |
| LCA17 | GDF6 | 8q22 |
| LCA18 | PRPH2 | 6p21 |

Retinal pigment epithelium-specific 65 kDa protein (RPE65), also referred to as retinoid isomerohydrolase, belongs to the carotenoid oxygenases family, is an enzyme in the visual cycle of vertebrates, and is encoded by the RPE65 gene in humans.

RPE65 is mainly expressed in retinal pigment epithelium (RPE) cells, and is also present in rod cells and cone cells. It is responsible for converting all-trans-retinyl esters into 11-cis-retinol during the phototransduction process. And then under the action of other enzymes, 11-cis-retinol is oxidized to 11-cis-retinal which in turn compounded with opsin to form active visual pigment, so as to activate the phototransduction pathway for detecting light by the brain.

The functional defect of RPE65 can result in LCA2, which accounts for about 6% to 16% of all LCA cases. Studies have shown that supplementing ocular cells having RPE65 functional defects with RPE65 with normal functionality can improve LCA.

### Recombinant AAV Vectors

Adeno-associated virus (AAV) belongs to the Parvoviridae family and is a single-stranded DNA (ssDNA) virus. The AAV genome is approximately 4.7 kilobases in length, and can comprise inverted terminal repeats (ITRs) at both ends of the DNA strand and two open reading frames (ORF) called rep and cap.

The “AAV inverted terminal repeat (ITR)” sequences can be sequences of about 145 nucleotides that exists at both ends of the natural single-stranded AAV genome. ITRs are symmetric nucleic acid sequences used for efficient replication in the adeno-associated virus genome, which can be used as a replication origin for viral DNA synthesis and can be necessary structural components of recombinant AAV vectors.

“Rep” can comprise the polynucleotide sequences encoding four rep proteins rep78, rep68, rep52 and rep40 required for the life cycle of AAV. “Cap” can comprise the polynucleotide sequences encoding AAV capsid proteins VP1, VP2, and VP3, wherein AAV capsid proteins VP1, VP2, and VP3 can interact to form an icositetrahedral symmetric AAV capsid.

AAV can effectively infect dividing and/or non-dividing human cells, and its genome can be integrated into a single chromosomal site in the host cell genome. Most importantly, although AAV exists in many people's bodies, current research believes that AAV is not related to any disease. Based on its high safety, low immunogenicity, wide host range, and ability to mediate long-term stable expression of exogenous genes in animals, AAV has become the most promising vector system in gene therapy.

To date, 13 different AAVs have been identified according to the difference of AAV serotypes or infected tissues or cells, namely AAV1-AAV13. And, as shown in Table 2 below, different AAVs have been developed as advantageous vector systems for transfection of specific cell types. Among the many AAV serotypes, serotype 2 (AAV2) is the most widely studied and used one, which can infect retinal epithelium, photoreceptor cells, skeletal muscle, central nervous system and liver cells, etc., and has been used as a vector for many clinical studies in progress.

TABLE 2
AAV serotypes and the tissues in which they are
used as vectors for delivery in gene therapy

| AAV serotypes | Tissues of delivery |
|---|---|
| AAV1, AAV2, AAV4, AAV5, AAV8, AAV9 | central nervous system |
| AAV1, AAV8, AAV9 | heart |
| AAV2 | kidney |
| AAV7, AAV8, AAV9 | liver |
| AAV4, AAV5, AAV6, AAV9 | lung |
| AAV8 | pancreas |
| AAV2, AAV5, AAV8 | photoreceptor cells |
| AAV1, AAV2, AAV4, AAV5, AAV8 | retinal epithelium |
| AAV1, AAV6, AAV7, AAV8, AAV9 | skeletal muscle |

As used herein, the term “recombinant AAV vectors (rAAV vectors)” refers to polynucleotide vectors containing one or more heterologous sequences (i.e., non-AAV-derived nucleic acid sequences) flanked by two AAV inverted terminal repeat sequences (ITRs). When present in host cells expressing AAV rep and cap proteins, the rAAV vectors can be replicated and packaged into AAV virus particles.

“Recombinant AAV (rAAV) virus” or “rAAV virus particle” refers to an AAV virus particle composed of an rAAV vector encapsulated by at least one AAV capsid protein. The host cells currently used for the production of rAAV virus particles can be cell types derived from mammals, such as 293 cells, COS cells, HeLa cells, KB cells, and other mammalian cell lines, as well as insect cells. The rAAV virus particles can be produced in the mammalian cell culture systems by providing rAAV plasmids. However, the output of most of the above mammalian cell culture systems is difficult to meet the requirements of clinical trials and commercial scale production. The rAAV virus particle production systems using insect cells such as Sf9 cells have recently been developed as well. However, to produce AAV in insect cells, some modifications must be made to obtain the correct stoichiometric ratio of AAV capsid proteins.

Baculovirus belongs to the Baculoviridae family and is a double-stranded circular DNA virus with a genome size between 90 kb and 230 kb. Baculoviruses are parasitic exclusively in arthropods and are known to infect more than 600 kinds of insects. In 1983, Smith et al. used Autographa Californica Multicapsid Nuclear Polyhedrosis Virus (AcMNPV) to successfully express human β-interferon in the Spodoptera frugiperda cell line Sf9, and created for the first time a baculovirus expression system (Mol Cell Biol, 1983, 3: 2156-2165). Since then, the baculovirus expression system has been continuously improved and developed, and has become a very widely used eukaryotic expression system. In 2002, Urabe et al. confirmed that Sf9 insect cells infected with baculovirus can support AAV replication. They used three recombinant baculoviruses carrying AAV's rep gene, Cap gene and ITR core expression elements, respectively, to co-infect Sf9 cells, and successfully prepared rAAV virus particles. On this basis, researchers have successively developed systems that are more suitable for large-scale preparation of rAAV virus particles.

At present, there are mainly two methods for large-scale preparation of rAAV virus particles using baculovirus expression systems: the Two Bac system and the One Bac system that relies on packaging cell lines. The main process of using the Two Bac system to prepare rAAV virus particles is to integrate the AAV rep gene and cap gene into a baculovirus genome, integrate the ITR core expression elements and the target gene of interest into another baculovirus genome, and then co-infect host cells using the two recombinant baculoviruses described above to produce rAAV virus particles carrying the target gene. The main process of using the One Bac system that relies on packaging cell lines to prepare rAAV virus particles is to first establish a packaging cell line that induces the expression of rep gene and cap gene. This packaging cell line integrates expression elements for rep gene and cap gene, wherein the rep gene and the cap gene are placed under the regulation of the baculovirus late gene expression strong promoter polyhedrin (polh) and/or p10, respectively, and in addition to the rep and cap, hr2 enhancer sequence and/or AAV's rep protein binding sequence are further added. After being infected with a recombinant baculovirus containing AAV ITR and the target gene, the rep gene and cap gene in the packaging cell line are induced to express, resulting in rAAV virus particles integrated with the target gene.

In some embodiments, the rAAV vectors used to carry target genes in the rAAV virus particles may also include one or more “expression regulatory elements”. As used herein, the term “expression regulatory elements” refers to nucleic acid sequences that affect the expression of operably linked polynucleotides, including polynucleotide sequences that promote the transcription and translation of heterologous polynucleotides. The expression regulatory elements that can be used in the present disclosure include but are not limited to promoters, enhancers, intron splicing signals, poly(A), inverted terminal repeats (ITR) and the like.

A “promoter” is a DNA sequence located adjacent to a heterologous polynucleotide sequence encoding a target product, and is usually operably linked to an adjacent sequence, such as a heterologous polynucleotide. A promoter generally increases the amount of heterologous polynucleotide expressed compared to that without the promoter.

An “enhancer” is a sequence that enhances the activity of a promoter. Unlike a promoter, an enhancer does not have promoter activity, and usually can function independently of its position relative to the promoter (i.e., upstream or downstream of the promoter). Non-limiting examples of enhancer elements (or portions thereof) that can be used in the present disclosure include baculovirus enhancers and enhancer elements found in insect cells.

A “stuffer sequence” refers to a nucleotide sequence contained in a larger nucleic acid molecule (such as a vector), and is usually used to produce a desired spacing between two nucleic acid features (such as between a promoter and a coding sequence), or extend a nucleic acid molecule to a desired length. The stuffer sequence does not contain protein coding information, and may have unknown/synthetic origin and/or is unrelated to other nucleic acid sequences within the larger nucleic acid molecule.

### Codon Optimization

There are 20 amino acids that make up a protein, and 64 codons that encode amino acids. Each amino acid corresponds to at least one codon, and one amino acid can correspond to up to 6 codons (degenerate codons). Different organisms, even different protein-coding genes of the same organism, have different frequency of use of degenerate codons and have a certain preference. Among them, codons with high frequency are called preferred codons, and those that are rarely used are called rare or low-frequency codons. Optimization of gene codons can increase protein expression level by utilizing preferred codons, avoiding rare or low-frequency codons with low utilization, simplifying the secondary structure of mRNA after gene transcription, icorporating motifs that are conducive to high-efficiency expression and reducing motifs that are unfavorable to expression, and adjusting GC content, and the like. Although there are many general codon optimization principles, these general optimization principles cannot be uniformly applied to a single gene therapy vector. Different general optimization principles may contradict each other. For example, changing the composition of CpG islands or the GC content of the coding region may affect the choice of codon usage preference. In addition, different codon optimizations may lead to different post-translational modifications and different biological activities.

The present disclosure provides a nucleotide sequence encoding RPE65 polypeptide. In some embodiments, the nucleotide sequence is codon-optimized. After codon optimization, the nucleotide sequence contains an altered number of CpG dinucleotides compared to the wild type RPE65 nucleotide sequence. In some embodiments, the sequence encoding RPE65 described herein comprises about 95% of CpG dinucleotides as compared to the wild type RPE65 nucleotide sequence. In some embodiments, the sequence encoding RPE65 described herein comprises about 90% of CpG dinucleotides as compared to the wild type RPE65 nucleotide sequence. In some embodiments, the sequence encoding RPE65 described herein comprises about 80% of CpG dinucleotides as compared to the wild type RPE65 nucleotide sequence. In some embodiments, the sequence encoding RPE65 described herein comprises about 70% of CpG dinucleotides as compared to the wild type RPE65 nucleotide sequence. In some embodiments, the sequence encoding RPE65 described herein comprises about 60% of CpG dinucleotides as compared to the wild type RPE65 nucleotide sequence. In some embodiments, the sequence encoding RPE65 described herein comprises about 50% of CpG dinucleotides as compared to the wild type RPE65 nucleotide sequence. In some embodiments, the sequence encoding RPE65 described herein comprises about 40% of CpG dinucleotides as compared to the wild type RPE65 nucleotide sequence. In some embodiments, the sequence encoding RPE65 described herein comprises about 30% of CpG dinucleotides as compared to the wild type RPE65 nucleotide sequence. In some embodiments, the sequence encoding RPE65 described herein comprises about 20% of CpG dinucleotides as compared to the wild type RPE65 nucleotide sequence. In some embodiments, the sequence encoding RPE65 described herein comprises about 10% or less of CpG dinucleotides as compared to the wild type RPE65 nucleotide sequence. In some embodiments, the sequence encoding RPE65 described herein comprises at most about 10%, 20%, 30%, 40%, 50%, 60%, 70%, 80% or 90% of CpG dinucleotides as compared to the wild type RPE65 nucleotide sequence. In some embodiments, the sequence encoding RPE65 described herein comprises at most about 60% of CpG dinucleotides as compared to the wild type RPE65 nucleotide sequence. In some embodiments, the sequence encoding RPE65 described herein comprises at most about 50% of CpG dinucleotides as compared to the wild type RPE65 nucleotide sequence.

In some embodiments, the sequence encoding RPE65 described herein comprises no more than 20 CpG dinucleotides. In some embodiments, the sequence encoding RPE65 described herein comprises no more than 19 CpG dinucleotides. In some embodiments, the sequence encoding RPE65 described herein comprises no more than 18 CpG dinucleotides. In some embodiments, the sequence encoding RPE65 described herein comprises no more than 17 CpG dinucleotides. In some embodiments, the sequence encoding RPE65 described herein comprises no more than 16 CpG dinucleotides. In some embodiments, the sequence encoding RPE65 described herein comprises no more than 15 CpG dinucleotides. In some embodiments, the sequence encoding RPE65 described herein comprises no more than 14 CpG dinucleotides. In some embodiments, the sequence encoding RPE65 described herein comprises no more than 13 CpG dinucleotides. In some embodiments, the sequence encoding RPE65 described herein comprises no more than 12 CpG dinucleotides. In some embodiments, the sequence encoding RPE65 described herein comprises no more than 11 CpG dinucleotides. In some embodiments, the sequence encoding RPE65 described herein comprises no more than 10 CpG dinucleotides. In some embodiments, the sequence encoding RPE65 described herein comprises no more than 9 CpG dinucleotides. In some embodiments, the sequence encoding RPE65 described herein comprises no more than 8 CpG dinucleotides. In some embodiments, the sequence encoding RPE65 described herein comprises no more than 7 CpG dinucleotides. In some embodiments, the sequence encoding RPE65 described herein comprises no more than 6 CpG dinucleotides. In some embodiments, the sequence encoding RPE65 described herein comprises no more than 5 CpG dinucleotides. In some embodiments, the sequence encoding RPE65 described herein comprises no more than 4 CpG dinucleotides. In some embodiments, the sequence encoding RPE65 described herein comprises no more than 3 CpG dinucleotides. In some embodiments, the sequence encoding RPE65 described herein comprises no more than 2 CpG dinucleotides. In some embodiments, the sequence encoding RPE65 described herein comprises no more than 1 CpG dinucleotides. In some embodiments, the sequence encoding RPE65 described herein does not comprise CpG dinucleotides. In some embodiments, the sequence encoding RPE65 described herein comprises 5 to 20 CpG dinucleotides. In some embodiments, the sequence encoding RPE65 described herein comprises 5 to 15 CpG dinucleotides. In some embodiments, the sequence encoding RPE65 described herein comprises 12 to 20 CpG dinucleotides. In some embodiments, the sequence encoding RPE65 described herein comprises 2 to 10 CpG dinucleotides. In some embodiments, the sequence encoding RPE65 described herein comprises 0 to 5 CpG dinucleotides.

In some embodiments, the sequence encoding RPE65 described herein comprises an increased number of CpG dinucleotides as compared to the wild type RPE65 nucleotide sequence. In some embodiments, the sequence encoding RPE65 described herein comprises about 200% of CpG dinucleotides as compared to the wild type RPE65 nucleotide sequence. In some embodiments, the sequence encoding RPE65 described herein comprises about 300% of CpG dinucleotides as compared to the wild type RPE65 nucleotide sequence. In some embodiments, the sequence encoding RPE65 described herein comprises about 400% of CpG dinucleotides as compared to the wild type RPE65 nucleotide sequence. In some embodiments, the sequence encoding RPE65 described herein comprises about 500% of CpG dinucleotides as compared to the wild type RPE65 nucleotide sequence. In some embodiments, the sequence encoding RPE65 described herein comprises about 600% of CpG dinucleotides as compared to the wild type RPE65 nucleotide sequence. In some embodiments, the sequence encoding RPE65 described herein comprises about 700% of CpG dinucleotides as compared to the wild type RPE65 nucleotide sequence.

In some embodiments, the sequence encoding RPE65 described herein comprises no less than 50 CpG dinucleotides. In some embodiments, the sequence encoding RPE65 described herein comprises no less than 100 CpG dinucleotides. In some embodiments, the sequence encoding RPE65 described herein comprises no less than 150 CpG dinucleotides. In some embodiments, the sequence encoding RPE65 described herein comprises no less than 200 CpG dinucleotides. In some embodiments, the sequence encoding RPE65 described herein comprises no less than 250 CpG dinucleotides. In some embodiments, the sequence encoding RPE65 described herein comprises no less than 300 CpG dinucleotides. In some embodiments, the sequence encoding RPE65 described herein comprises about 50 to 300 CpG dinucleotides. In some embodiments, the sequence encoding RPE65 described herein comprises about 100 to 250 CpG dinucleotides. In some embodiments, the sequence encoding RPE65 described herein comprises about 150 to 200 CpG dinucleotides. In some embodiments, the sequence encoding RPE65 described herein comprises about 150 CpG dinucleotides. In some embodiments, the sequence encoding RPE65 described herein comprises about 100 CpG dinucleotides.

In some embodiments, the sequence encoding RPE65 described herein comprises a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In some embodiments, the coding sequence comprises SEQ ID NO: 2. In some embodiments, the coding sequence has at least 80% identity to SEQ ID No: 2. In some embodiments, the coding sequence has at least 90% identity to SEQ ID No: 2. In some embodiments, the coding sequence has at least 95% identity to SEQ ID No: 2. In some embodiments, the coding sequence comprises SEQ ID NO: 3. In some embodiments, the coding sequence has at least 80% identity to SEQ ID No: 3. In some embodiments, the coding sequence has at least 90% identity to SEQ ID No: 3. In some embodiments, the coding sequence has at least 95% identity to SEQ ID No: 3. In some embodiments, the coding sequence has at least 98% identity to SEQ ID No: 3. In some embodiments, the coding sequence comprises SEQ ID NO: 4. In some embodiments, the coding sequence has at least 80% identity to SEQ ID No: 4. In some embodiments, the coding sequence has at least 90% identity to SEQ ID No: 4. In some embodiments, the coding sequence has at least 95% identity to SEQ ID No: 4. In some embodiments, the coding sequence comprises SEQ ID NO: 5. In some embodiments, the coding sequence has at least 80% identity to SEQ ID No: 5. In some embodiments, the coding sequence has at least 90% identity to SEQ ID No: 5. In some embodiments, the coding sequence has at least 95% identity to SEQ ID No: 5. In some embodiments, the coding sequence comprises SEQ ID NO: 6. In some embodiments, the coding sequence has at least 80% identity to SEQ ID No: 6. In some embodiments, the coding sequence has at least 90% identity to SEQ ID No: 6. In some embodiments, the coding sequence has at least 95% identity to SEQ ID No: 6. In some embodiments, the coding sequence has at least 98% identity to SEQ ID No: 6. In some embodiments, the coding sequence comprises SEQ ID NO: 7. In some embodiments, the coding sequence has at least 80% identity to SEQ ID No: 7. In some embodiments, the coding sequence has at least 90% identity to SEQ ID No: 7. In some embodiments, the coding sequence has at least 95% identity to SEQ ID No: 7. In some embodiments, the coding sequence comprises SEQ ID NO: 8. In some embodiments, the coding sequence has at least 80% identity to SEQ ID No: 8. In some embodiments, the coding sequence has at least 90% identity to SEQ ID No: 8. In some embodiments, the coding sequence has at least 95% identity to SEQ ID No: 8. In some embodiments, the coding sequence comprises SEQ ID NO: 9. In some embodiments, the coding sequence has at least 80% identity to SEQ ID No: 9. In some embodiments, the coding sequence has at least 90% identity to SEQ ID No: 9. In some embodiments, the coding sequence has at least 95% identity to SEQ ID No: 9. In some embodiments, the coding sequence comprises SEQ ID NO: 10. In some embodiments, the coding sequence has at least 80% identity to SEQ ID No: 10. In some embodiments, the coding sequence has at least 90% identity to SEQ ID No: 10. In some embodiments, the coding sequence has at least 95% identity to SEQ ID No: 10.

In some embodiments, the nucleotide sequence encoding the adeno-associated virus (AAV) capsid protein is codon-optimized. After codon optimization, the nucleotide sequence contains an altered number of CpG dinucleotides compared to the wild type AAV capsid protein nucleotide sequence. In some embodiments, the nucleotide sequence encoding the adeno-associated virus (AAV) rep protein is codon-optimized. After codon optimization, the nucleotide sequence contains an altered number of CpG dinucleotides compared to the wild type AAV rep protein nucleotide sequence.

### Composition

In one aspect, the present disclosure provides a composition comprising: (i) a first polynucleotide encoding an adeno-associated virus (AAV) protein, and (ii) a second polynucleotide comprising a sequence encoding a RPE65 polypeptide. In one aspect, the present disclosure provides a composition comprising: (i) a first polynucleotide encoding an adeno-associated virus (AAV) protein, and (ii) a second polynucleotide comprising a sequence encoding a RPE65 polypeptide, wherein the sequence is codon-optimized and contains an altered number of CpG dinucleotides as compared to a wild type RPE65 nucleotide sequence.

The RPE65 polypeptides described herein may be RPE65 derived from any mammal and variants thereof. In some embodiments, the mammal includes, but is not limited to, primates (e.g., humans), bovines, canines, felines, and rodents (e.g., guinea pigs, rats, or mice). In some embodiments, the RPE65 polypeptides described herein are human-derived RPE65 or variants thereof. In some embodiments, the RPE65 polypeptides described herein comprise a sequence having at least 75% identity to human RPE65. In some embodiments, the RPE65 polypeptides described herein comprise a sequence having at least 80% identity to human RPE65. In some embodiments, the RPE65 polypeptides described herein comprise a sequence having at least 85% identity to human RPE65. In some embodiments, the RPE65 polypeptides described herein comprise a sequence having at least 90% identity to human RPE65. In some embodiments, the RPE65 polypeptides described herein comprise a sequence having at least 95% identity to human RPE65. In some embodiments, the RPE65 polypeptides described herein comprise a sequence having at least 96% identity to human RPE65. In some embodiments, the RPE65 polypeptides described herein comprise a sequence having at least 97% identity to human RPE65. In some embodiments, the RPE65 polypeptides described herein comprise a sequence having at least 98% identity to human RPE65. In some embodiments, the RPE65 polypeptides described herein comprise a sequence having at least 99% identity to human RPE65. In some embodiments, the RPE65 polypeptides described herein comprise a sequence that has one or more amino acid mutations, substitutions, deletions, or additions compared to human RPE65.

A composition described herein can comprise a polynucleotide that comprises a sequence encoding a RPE65 polypeptide. In some embodiments, the RPE65 polypeptides described herein comprise the sequence of SEQ ID No: 1. In some embodiments, the RPE65 polypeptides described herein comprise a sequence having at least 75% identity to SEQ ID No: 1. In some embodiments, the RPE65 polypeptides described herein comprise a sequence having at least 80% identity to SEQ ID No: 1. In some embodiments, the RPE65 polypeptides described herein comprise a sequence having at least 85% identity to SEQ ID No: 1. In some embodiments, the RPE65 polypeptides described herein comprise a sequence having at least 90% identity to SEQ ID No: 1. In some embodiments, the RPE65 polypeptides described herein comprise a sequence having at least 95% identity to SEQ ID No: 1. In some embodiments, the RPE65 polypeptides described herein comprise a sequence having at least 96% identity to SEQ ID No: 1. In some embodiments, the RPE65 polypeptides described herein comprise a sequence having at least 97% identity to SEQ ID No: 1. In some embodiments, the RPE65 polypeptides described herein comprise a sequence having at least 98% identity to SEQ ID No: 1. In some embodiments, the RPE65 polypeptides described herein comprise a sequence having at least 99% identity to SEQ ID No: 1. In some embodiments, the RPE65 polypeptides described herein comprise a sequence that has one or more amino acid mutations, substitutions, deletions, or additions compared to SEQ ID No: 1.

In some embodiments, provided herein is a polynucleotide that comprises a sequence encoding a RPE65 polypeptide, wherein the sequence comprises a reduced number of CpG dinucleotides as compared to the corresponding wild type RPE65 nucleotide sequence. In some embodiments, the sequence that encodes the RPE65 polypeptide comprises about 90% of CpG dinucleotides as compared to the wild type RPE65 nucleotide sequence. In some embodiments, the sequence comprises about 80% of CpG dinucleotides as compared to the wild type RPE65 nucleotide sequence. In some embodiments, the sequence comprises about 70% of CpG dinucleotides as compared to the wild type RPE65 nucleotide sequence. In some embodiments, the sequence comprises about 60% of CpG dinucleotides as compared to the wild type RPE65 nucleotide sequence. In some embodiments, the sequence comprises about 50% of CpG dinucleotides as compared to the wild type RPE65 nucleotide sequence. In some embodiments, the sequence comprises about 40% of CpG dinucleotides as compared to the wild type RPE65 nucleotide sequence. In some embodiments, the sequence comprises about 30% of CpG dinucleotides as compared to the wild type RPE65 nucleotide sequence. In some embodiments, the sequence comprises about 20% of CpG dinucleotides as compared to the wild type RPE65 nucleotide sequence. In some embodiments, the sequence comprises about 10% of CpG dinucleotides as compared to the wild type RPE65 nucleotide sequence.

In some embodiments, the sequence comprises no more than 20 CpG dinucleotides. In some embodiments, the sequence comprises no more than 19 CpG dinucleotides. In some embodiments, the sequence comprises no more than 18 CpG dinucleotides. In some embodiments, the sequence comprises no more than 17 CpG dinucleotides. In some embodiments, the sequence comprises no more than 16 CpG dinucleotides. In some embodiments, the sequence comprises no more than 15 CpG dinucleotides. In some embodiments, the sequence comprises no more than 14 CpG dinucleotides. In some embodiments, the sequence comprises no more than 13 CpG dinucleotides. In some embodiments, the sequence comprises no more than 12 CpG dinucleotides. In some embodiments, the sequence comprises no more than 11 CpG dinucleotides. In some embodiments, the sequence comprises no more than 10 CpG dinucleotides. In some embodiments, the sequence comprises no more than 9 CpG dinucleotides. In some embodiments, the sequence comprises no more than 8 CpG dinucleotides. In some embodiments, the sequence comprises no more than 7 CpG dinucleotides. In some embodiments, the sequence comprises no more than 6 CpG dinucleotides. In some embodiments, the sequence comprises no more than 5 CpG dinucleotides. In some embodiments, the sequence comprises no more than 4 CpG dinucleotides. In some embodiments, the sequence comprises no more than 3 CpG dinucleotides. In some embodiments, the sequence comprises no more than 2 CpG dinucleotides. In some embodiments, the sequence comprises no more than 1 CpG dinucleotides. In some embodiments, the sequence does not comprise CpG dinucleotides.

In some embodiments, the sequence comprises an increased number of CpG dinucleotides as compared to the wild type RPE65 nucleotide sequence. In some embodiments, the sequence comprises about 200% of CpG dinucleotides as compared to the wild type RPE65 nucleotide sequence. In some embodiments, the sequence comprises about 300% of CpG dinucleotides as compared to the wild type RPE65 nucleotide sequence. In some embodiments, the sequence comprises about 400% of CpG dinucleotides as compared to the wild type RPE65 nucleotide sequence. In some embodiments, the sequence comprises about 500% of CpG dinucleotides as compared to the wild type RPE65 nucleotide sequence. In some embodiments, the sequence comprises about 600% of CpG dinucleotides as compared to the wild type RPE65 nucleotide sequence. In some embodiments, the sequence comprises about 700% of CpG dinucleotides as compared to the wild type RPE65 nucleotide sequence.

In some embodiments, the sequence comprises no less than 50 CpG dinucleotides. In some embodiments, the sequence comprises no less than 100 CpG dinucleotides. In some embodiments, the sequence comprises no less than 150 CpG dinucleotides. In some embodiments, the sequence comprises no less than 200 CpG dinucleotides. In some embodiments, the sequence comprises no less than 250 CpG dinucleotides. In some embodiments, the sequence comprises no less than 300 CpG dinucleotides. In some embodiments, the sequence comprises about 50 to 300 CpG dinucleotides. In some embodiments, the sequence comprises about 100 to 250 CpG dinucleotides. In some embodiments, the sequence comprises about 150 to 200 CpG dinucleotides. In some embodiments, the sequence comprises about 150 CpG dinucleotides. In some embodiments, the sequence comprises about 100 CpG dinucleotides.

In some embodiments, the sequence is selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In some embodiments, the coding sequence comprises or is SEQ ID NO: 2. In some embodiments, the coding sequence has at least 80% identity to SEQ ID No: 2. In some embodiments, the coding sequence has at least 90% identity to SEQ ID No: 2. In some embodiments, the coding sequence has at least 95% identity to SEQ ID No: 2. In some embodiments, the coding sequence comprises or is SEQ ID NO: 3. In some embodiments, the coding sequence has at least 80% identity to SEQ ID No: 3. In some embodiments, the coding sequence has at least 90% identity to SEQ ID No: 3. In some embodiments, the coding sequence has at least 95% identity to SEQ ID No: 3. In some embodiments, the coding sequence comprises or is SEQ ID NO: 4. In some embodiments, the coding sequence has at least 80% identity to SEQ ID No: 4. In some embodiments, the coding sequence has at least 90% identity to SEQ ID No: 4. In some embodiments, the coding sequence has at least 95% identity to SEQ ID No: 4. In some embodiments, the coding sequence comprises or is SEQ ID NO: 5. In some embodiments, the coding sequence has at least 80% identity to SEQ ID No: 5. In some embodiments, the coding sequence has at least 90% identity to SEQ ID No: 5. In some embodiments, the coding sequence has at least 95% identity to SEQ ID No: 5. In some embodiments, the coding sequence comprises or is SEQ ID NO: 6. In some embodiments, the coding sequence has at least 80% identity to SEQ ID No: 6. In some embodiments, the coding sequence has at least 90% identity to SEQ ID No: 6. In some embodiments, the coding sequence has at least 95% identity to SEQ ID No: 6. In some embodiments, the coding sequence comprises or is SEQ ID NO: 7. In some embodiments, the coding sequence has at least 80% identity to SEQ ID No: 7. In some embodiments, the coding sequence has at least 90% identity to SEQ ID No: 7. In some embodiments, the coding sequence has at least 95% identity to SEQ ID No: 7. In some embodiments, the coding sequence comprises or is SEQ ID NO: 8. In some embodiments, the coding sequence has at least 80% identity to SEQ ID No: 8. In some embodiments, the coding sequence has at least 90% identity to SEQ ID No: 8. In some embodiments, the coding sequence has at least 95% identity to SEQ ID No: 8. In some embodiments, the coding sequence comprises or is SEQ ID NO: 9. In some embodiments, the coding sequence has at least 80% identity to SEQ ID No: 9. In some embodiments, the coding sequence has at least 90% identity to SEQ ID No: 9. In some embodiments, the coding sequence has at least 95% identity to SEQ ID No: 9. In some embodiments, the coding sequence comprises or is SEQ ID NO: 10. In some embodiments, the coding sequence has at least 80% identity to SEQ ID No: 10. In some embodiments, the coding sequence has at least 90% identity to SEQ ID No: 10. In some embodiments, the coding sequence has at least 95% identity to SEQ ID No: 10.

In some embodiments, the adeno-associated virus (AAV) protein may be from any AAV serotype. In some embodiments, the AAV protein may be from AAV serotype 1 (AAV1), AAV serotype 2 (AAV2), AAV2 variants (such as AAV2.7m8, AAV2(quad Y-F), and AAV2tYF), AAV serotype 3 (AAV3, including serotypes 3A and 3B), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 12 (AAV12), AAV serotype 13 (AAV13), AAV-Rh10, AAV-Rh74, AAV-2i8 and any other AAVs known. In some embodiments, the AAV protein has at least 75%, 80%, 85%, 90%, 95% or more identity to the wild type AAV proteins derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the AAV protein has one or more amino acid substitutions, deletions and/or additions compared to the wild type AAV proteins derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the AAV protein is from serotype AAV2 or variants thereof, serotype AAV5 or variants thereof, or serotype AAV8 or variants thereof.

In some embodiments, the AAV protein comprises a cap protein. In some embodiments, the first polynucleotide comprises a sequence encoding a cap protein. In some embodiments, the cap protein may be any structural protein known in the art that is capable of forming a functional AAV capsid (i.e., capable of packaging DNA and infecting target cells). In some embodiments, the cap protein comprises VP1, VP2, and VP3. In some embodiments, the cap protein needs not comprise all of VP1, VP2, and VP3, as long as it can produce a functional AAV capsid. In some embodiments, the cap protein comprises VP1 and VP2. In some embodiments, the cap protein comprises VP1 and VP3. In some embodiments, the cap protein comprises VP2 and VP3. In some embodiments, the cap protein comprises VP1. In some embodiments, the cap protein comprises VP2. In some embodiments, the cap protein comprises VP3.

The VP1, VP2, and VP3 may be derived from any AAV serotype. In some embodiments, the VP1 may be derived from AAV serotype 1 (AAV1), AAV serotype 2 (AAV2), AAV serotype 2 variants (such as AAV2.7m8, AAV2(quad Y-F), and AAV2tYF), AAV serotype 3 (AAV3, including serotypes 3A and 3B), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 12 (AAV12), AAV serotype 13 (AAV13), AAV-Rh10, AAV-Rh74, AAV-2i8 and any other AAVs known. In some embodiments, the VP1 has at least 75%, 80%, 85%, 90%, 95% or more identity to the wild type VP1s derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the VP1 has one or more amino acid substitutions, deletions and/or additions compared to the wild type VP1s derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8.

In some embodiments, the VP2 may be derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known. In some embodiments, the VP2 has at least 75%, 80%, 85%, 90%, 95% or more identity to the wild type VP2s derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the VP2 has one or more amino acid substitutions, deletions and/or additions compared to the wild type VP2s derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8.

In some embodiments, the VP3 may be derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known. In some embodiments, the VP3 has at least 75%, 80%, 85%, 90%, 95% or more identity to the wild type VP3s derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the VP3 has one or more amino acid substitutions, deletions and/or additions compared to the wild type VP3s derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8.

In some embodiments, the cap comprises VP1, VP2 and/or VP3 derived from the same serotype of AAV, for example, the cap may comprise VP1, VP2 and/or VP3 derived from AAV2. In some embodiments, the cap comprises VP1, VP2 and/or VP3 derived from different serotypes of AAV, for example, the cap may comprise VP1, VP2 and/or VP3 derived from any one or more of AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 and AAV-2i8.

In some embodiments, the sequence encoding the cap protein is operably linked to a promoter. The promoter may be any suitable promoter known in the art that can drive the expression of the cap. In some embodiments, the promoter may be a tissue-specific promoter, a constitutive promoter, or a regulatable promoter. In some embodiments, the promoter may be selected from different sources, for example, the promoter can be a viral promoter, a plant promoter, and a mammalian promoter.

Examples of the promoter include, but are not limited to, human cytomegalovirus (CMV) immediate-early enhancer/promoter, SV40 early enhancer/promoter, JC polyomavirus promoter, myelin basic protein (MBP) or glial fibrillary acidic protein (GFAP) promoter, herpes simplex virus (HSV-1) latency-related promoter (LAP), Rous sarcoma virus (RSV) long terminal repeat (LTR) promoter, neuron-specific promoter (NSE), platelet-derived growth factor (PDGF) promoter, hSYN, melanin-concentrating hormone (MCH) promoter, CBA, matrix metalloprotein promoter (MPP), chicken β-actin promoter, CAG, MNDU3, PGK and EF1a promoters.

In some embodiments, the promoter is a promoter suitable for expression in insect cells. In some embodiments, the promoter suitable for expression in insect cells include, but is not limited to, a polh promoter, a p10 promoter, a basic promoter, an inducible promoter, an E1 promoter or a ΔE1 promoter. In some embodiments, the promoter is a polh promoter. In some embodiments, the promoter is a p10 promoter.

In some embodiments, the 3′ end of the nucleotide sequence encoding the cap protein further comprises a polyadenylation sequence or a “poly(A) sequence”. In some embodiments, the length of the polyadenylation sequence or “poly(A) sequence” can range from about 1-500 bp. In some embodiments, the length of the polyadenylation sequence or “poly(A) sequence” can be, but is not limited to, 1, 2, 3, 4, 5, 6, 7, 8, 9, 10, 20, 30, 50, 100, 200, or 500 nucleotides.

In some embodiments, the AAV protein contained in the composition of the present disclosure further comprises an adeno-associated virus (AAV) rep protein. In some embodiments, the first polynucleotide comprises a sequence encoding an AAV rep protein, wherein the rep protein may be a replication protein necessary for any rAAV vector to replicate and package into rAAV virus particles. In some embodiments, the rep protein comprises rep78, rep68, rep52 and rep40. In some embodiments, the rep protein needs not comprise all of rep78, rep68, rep52 and rep40, as long as it can allow the rAAV vector to replicate and package into rAAV virus particles. In some embodiments, the rep protein comprises any three of rep78, rep68, rep52 and rep40. In some embodiments, the rep protein comprises any two of rep78, rep68, rep52 and rep40. In some embodiments, the rep protein comprises any one of rep78, rep68, rep52 and rep40. In some embodiments, the rep protein comprises rep78 and rep52. In some embodiments, the rep protein comprises rep78 and rep40. In some embodiments, the rep protein comprises rep68 and rep52. In some embodiments, the rep protein comprises rep68 and rep40.

The rep78, rep68, rep52 and rep40 may be derived from any AAV serotype. In some embodiments, the rep78 may be derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known. In some embodiments, the rep78 has at least 75%, 80%, 85%, 90%, 95% or more identity to the wild type rep78s derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the rep78 has one or more amino acid substitutions, deletions and/or additions compared to the wild type rep78s derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8.

In some embodiments, the rep68 may be derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known. In some embodiments, the rep68 has at least 75%, 80%, 85%, 90%, 95% or more identity to the wild type rep68s derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the rep68 has one or more amino acid substitutions, deletions and/or additions compared to the wild type rep68s derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8.

In some embodiments, the rep52 may be derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known. In some embodiments, the rep52 has at least 75%, 80%, 85%, 90%, 95% or more identity to the wild type rep52s derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the rep52 has one or more amino acid substitutions, deletions and/or additions compared to the wild type rep52s derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8.

In some embodiments, the rep40 may be derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known. In some embodiments, the rep40 has at least 75%, 80%, 85%, 90%, 95% or more identity to the wild type rep40s derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the rep40 has one or more amino acid substitutions, deletions and/or additions compared to the wild type rep40s derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8.

In some embodiments, the rep comprises rep78, rep68, rep52 and/or rep40 derived from the same serotype of AAV, for example, the rep may comprise rep78, rep68, rep52 and/or rep40 derived from AAV2. In some embodiments, the rep comprises rep78, rep68, rep52 and/or rep40 derived from different serotypes of AAV, for example, the rep may comprise rep78, rep68, rep52 and/or rep40 derived from any one or more of AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other known AAV.

In some embodiments, the sequence encoding the rep protein is operably linked to a promoter. The promoter can be any suitable promoter known in the art that can drive the expression of the rep. In some embodiments, the promoter can be a tissue-specific promoter, a constitutive promoter, or a regulatable promoter. In some embodiments, the promoter can be selected from different sources, for example, the promoter can be a viral promoter, a plant promoter, and a mammalian promoter.

Examples of the promoter include, but are not limited to, human cytomegalovirus (CMV) immediate-early enhancer/promoter, SV40 early enhancer/promoter, JC polyomavirus promoter, myelin basic protein (MBP) or glial fibrillary acidic protein (GFAP) promoter, herpes simplex virus (HSV-1) latency-related promoter (LAP), Rous sarcoma virus (RSV) long terminal repeat (LTR) promoter, neuron-specific promoter (NSE), platelet-derived growth factor (PDGF) promoter, hSYN, melanin-concentrating hormone (MCH) promoter, CBA, matrix metalloprotein promoter (MPP), chicken β-actin promoter, CAG, MNDU3, PGK and EF1 a promoters.

In some embodiments, the promoter is a promoter suitable for expression in insect cells. In some embodiments, the promoter suitable for expression in insect cells include, but is not limited to, a polh promoter, a p10 promoter, a basic promoter, an inducible promoter, an E1 promoter or a ΔE1 promoter. In some embodiments, the promoter is a polh promoter. In some embodiments, the promoter is a p10 promoter.

In some embodiments, the 3′ end of the nucleotide sequence encoding the rep protein further comprises a polyadenylation sequence or a “poly(A) sequence”. In some embodiments, the length of the polyadenylation sequence or “poly(A) sequence” may range from about 1-500 bp. In some embodiments, the length of the polyadenylation sequence or “poly(A) sequence” may be, but is not limited to, 1, 2, 3, 4, 5, 6, 7, 8, 9, 10, 20, 30, 50, 100, 200, or 500 nucleotides.

In some embodiments, the cap and the rep may be derived from the same AAV serotype. In some embodiments, the cap and the rep may be derived from the same AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 or any other AAVs known and variants.

In some embodiments, the cap and the rep may be derived from different AAV serotypes, for example, the cap and the rep may be derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 or any other AAVs known, respectively. For example, in some embodiments, the cap may be derived from AAV2 and the rep is derived from AAV5.

In some embodiments, the first polynucleotide is codon-optimized. In some embodiments, the coding sequence of the AAV protein is codon-optimized. In some embodiments, the coding sequence of the AAV cap protein is codon-optimized. In some embodiments, the coding sequence of the AAV rep protein is codon-optimized. In some embodiments, the coding sequence of the promoter is codon-optimized.

In some embodiments, the second polynucleotide comprises a promoter, and the promoter is operably linked to the sequence. In some embodiments, the promoter is CMV, CAG, MNDU3, PGK, EF1a, Ubc promoter or ocular tissue specific promoter. In some embodiments, the ocular tissue-specific promoter is selected from the RPE 65 gene promoter, human retinal binding protein (CRALBP) gene promoter, murine 11-cis-retinol dehydrogenase (RDH) gene promoter, rhodopsin promoter, rhodopsin kinase promoter, tissue inhibitor of metalloproteinase 3 (Timp3) promoter, photoreceptor retinol binding protein promoter and vitelliform macular dystrophy 2 promoter, or interphotoreceptor retinoid-binding protein (IRBP) promoters.

In some embodiments, the sequence further comprises a WPRE sequence at the 3′ end.

In some embodiments, the sequence further comprises a polyadenylation sequence or a “poly(A) sequence” at the 3′ end. In some embodiments, the length of the polyadenylation sequence or “poly(A) sequence” may range from about 1-500 bp. In some embodiments, the length of the polyadenylation sequence or “poly(A) sequence” may be, but is not limited to, 1, 2, 3, 4, 5, 6, 7, 8, 9, 10, 20, 30, 50, 100, 200, or 500 nucleotides. In some embodiments, the length of the poly(A) sequence is 5 to 100, 5 to 50, 10 to 50, 10 to 25, 25 to 50, or 25-75 nucleotides. In some embodiments, the poly(A) sequence is one of SV40pA, hGHpA and bGHpA.

In some embodiments, the second polynucleotide further comprises one or more other regulatory sequences. The regulatory sequences include, but are not limited to, inverted terminal repeats (ITR), enhancers, splicing signals, polyadenylation signals, stuffer sequences, terminators, protein degradation signals, internal ribosome entry elements (IRES), 2A sequences, and the like.

In some embodiments, the second polynucleotide further comprises an enhancer region. In some embodiments, the enhancer region comprises an SV40 enhancer, an immediate-early cytomegalovirus enhancer, an IRBP enhancer, and an enhancer derived from an immunoglobulin gene. In some embodiments, the enhancer region is located upstream of the CMV, CAG, MNDU3, PGK, and EF1a promoter. In some embodiments, the enhancer is located upstream of the ocular tissue-specific promoter. In some embodiments, the enhancer region is located downstream of the CMV, CAG, MNDU3, PGK, and EF1a promoter. In some embodiments, the enhancer is located downstream of the ocular tissue-specific promoter.

In some embodiments, the second polynucleotide further comprises an inverted terminal repeat (ITR) sequence. In some embodiments, the second polynucleotide comprises at least one inverted terminal repeat (ITR) sequence. In some embodiments, the second polynucleotide comprises two inverted terminal repeat sequences (ITRs). In some embodiments, the two ITRs are the same. In some embodiments, the two ITRs are different from each other. In some embodiments, the inverted terminal repeat sequences (ITRs) are ITRs derived from AAV. In some embodiments, the ITR may be derived from ITRs of AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known. In some embodiments, the ITR has one or more base mutations, insertions or deletions compared to wild type ITRs derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known, as long as it retains the desired function as a terminal repeat sequence, such as replication of the target gene, packaging and/or integration of virus particles, and the like.

In some embodiments, the second polynucleotide further comprises one or more stuffer sequences. In some embodiments, the stuffer sequence is located upstream of the CMV, CAG, MNDU3, PGK, and EF1a promoter sequence. In some embodiments, the stuffer sequence is located downstream of the CMV, CAG, MNDU3, PGK, and EF1a promoter sequence. In some embodiments, the stuffer sequence is located upstream of the ocular tissue-specific promoter. In some embodiments, the stuffer sequence is located downstream of the ocular tissue-specific promoter. In some embodiments, the stuffer sequence is located at the 5′ end of the 5′ ITR sequence. In some embodiments, the stuffer sequence is located at the 3′ end of the 5′ ITR sequence. In some embodiments, the stuffer sequence is located at the 5′ end of the 5′ ITR sequence. In some embodiments, the stuffer sequence is located at the 5′ end of the 3′ ITR sequence. In some embodiments, the stuffer sequence is located at the 3′ end of the 3′ ITR sequence.

In some embodiments, the length of the stuffer sequence may be about 0.1 kb-5 kb, such as, but are not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb or 5.0 kb.

In some embodiments, the second polynucleotide further comprises sequences encoding one or more other therapeutic protein. In some embodiments, the therapeutic protein is selected from the group consisting of: ATP-binding cassette sub-family A member 4 (ABCA4), retinol dehydrogenase 12 (RDH12), retinol dehydrogenase 8 (RDH8), retinol binding protein 3 (RBP 3), retinol binding protein 1 (RBP 1), lecithin retinol acyltransferase (LRAT), retinaldehyde binding protein 1 (Rlbp1), retinol dehydrogenase 10 (RDH10), and retinol dehydrogenase hydrogenase 11 (RDH11).

In some embodiments, the sequences encoding the other therapeutic proteins are linked to the sequence by a sequence encoding a linker. In some embodiments, the linker is a cleavable linker. In some embodiments, the cleavable linker comprises a sequence of a 2A peptide. In some embodiments, the 2A peptide may be selected from 2A peptides derived from aphthoviruses or cardioviruses, such as 2A peptides derived from foot-and-mouth disease virus (FMDV), equine rhinitis A virus (ERAV), Thoseaasigna virus (TaV) or porcine teschen virus (PTV-1).

In some embodiments, the second polynucleotide is codon-optimized. In some embodiments, the promoter is codon-optimized. In some embodiments, the stuffer sequence is codon-optimized. In some embodiments, the other therapeutic proteins are codon-optimized. In some embodiments, the linker sequence is codon-optimized.

In some embodiments, the second polynucleotide comprises no more than 500 CpG dinucleotides. In some embodiments, the second polynucleotide comprises no more than 450 CpG dinucleotides. In some embodiments, the second polynucleotide comprises no more than 400 CpG dinucleotides. In some embodiments, the second polynucleotide comprises no more than 350 CpG dinucleotides. In some embodiments, the second polynucleotide comprises no more than 300 CpG dinucleotides. In some embodiments, the second polynucleotide comprises no more than 250 CpG dinucleotides. In some embodiments, the second polynucleotide comprises no more than 200 CpG dinucleotides.

In some embodiments, the second polynucleotide comprises about 200 to 500 CpG dinucleotides. In some embodiments, the second polynucleotide comprises about 250 to 450 CpG dinucleotides. In some embodiments, the second polynucleotide comprises about 300 to 400 CpG dinucleotides. In some embodiments, the second polynucleotide comprises about 200 to 400 CpG dinucleotides. In some embodiments, the second polynucleotide comprises about 200 to 300 CpG dinucleotides. In some embodiments, the second polynucleotide comprises about 210 to 290 CpG dinucleotides. In some embodiments, the second polynucleotide comprises about 220 to 280 CpG dinucleotides. In some embodiments, the second polynucleotide comprises about 230 to 270 CpG dinucleotides. In some embodiments, the second polynucleotide comprises about 240 to 260 CpG dinucleotides. In some embodiments, the second polynucleotide comprises about 250 CpG dinucleotides.

### Recombinant AAV Virus Particles

In one aspect, the present disclosure provides a recombinant adeno-associated virus (rAAV) particle, comprising an expression cassette polynucleotide sequence that comprises a coding sequence of RPE65 polypeptide. In some embodiments, the coding sequence is codon-optimized and contains an altered number of CpG dinucleotides as compared to a wild type RPE65 nucleotide sequence.

In some embodiments, the coding sequence comprises a reduced number of CpG dinucleotides as compared to the wild type RPE65 nucleotide sequence. In some embodiments, the coding sequence comprises about 90% of CpG dinucleotides as compared to the wild type RPE65 nucleotide sequence. In some embodiments, the coding sequence comprises about 80% of CpG dinucleotides as compared to the wild type RPE65 nucleotide sequence. In some embodiments, the coding sequence comprises about 70% of CpG dinucleotides as compared to the wild type RPE65 nucleotide sequence. In some embodiments, the coding sequence comprises about 60% of CpG dinucleotides as compared to the wild type RPE65 nucleotide sequence. In some embodiments, the coding sequence comprises about 50% of CpG dinucleotides as compared to the wild type RPE65 nucleotide sequence. In some embodiments, the coding sequence comprises about 40% of CpG dinucleotides as compared to the wild type RPE65 nucleotide sequence. In some embodiments, the coding sequence comprises about 30% of CpG dinucleotides as compared to the wild type RPE65 nucleotide sequence. In some embodiments, the coding sequence comprises about 20% of CpG dinucleotides as compared to the wild type RPE65 nucleotide sequence. In some embodiments, the coding sequence comprises about 10% of CpG dinucleotides as compared to the wild type RPE65 nucleotide sequence.

In some embodiments, the coding sequence comprises no more than 25 CpG dinucleotides. In some embodiments, the coding sequence comprises no more than 20 CpG dinucleotides. In some embodiments, the coding sequence comprises no more than 19 CpG dinucleotides. In some embodiments, the coding sequence comprises no more than 18 CpG dinucleotides. In some embodiments, the coding sequence comprises no more than 17 CpG dinucleotides. In some embodiments, the coding sequence comprises no more than 16 CpG dinucleotides. In some embodiments, the coding sequence comprises no more than 15 CpG dinucleotides. In some embodiments, the coding sequence comprises no more than 14 CpG dinucleotides. In some embodiments, the coding sequence comprises no more than 13 CpG dinucleotides. In some embodiments, the coding sequence comprises no more than 12 CpG dinucleotides. In some embodiments, the coding sequence comprises no more than 11 CpG dinucleotides. In some embodiments, the coding sequence comprises no more than 10 CpG dinucleotides. In some embodiments, the coding sequence comprises no more than 9 CpG dinucleotides. In some embodiments, the coding sequence comprises no more than 8 CpG dinucleotides. In some embodiments, the coding sequence comprises no more than 7 CpG dinucleotides. In some embodiments, the coding sequence comprises no more than 6 CpG dinucleotides. In some embodiments, the coding sequence comprises no more than 5 CpG dinucleotides. In some embodiments, the coding sequence comprises no more than 4 CpG dinucleotides. In some embodiments, the coding sequence comprises no more than 3 CpG dinucleotides. In some embodiments, the coding sequence comprises no more than 2 CpG dinucleotides. In some embodiments, the coding sequence comprises no more than 1 CpG dinucleotides. In some embodiments, the coding sequence does not comprise CpG dinucleotides. In some embodiments, the coding sequence comprises at least 1, 2, 3, 4, 5, or 10 CpG dinucleotides. In some embodiments, the coding sequence comprises 5 to 15 CpG dinucleotides. In some embodiments, the coding sequence comprises 7 to 12 CpG dinucleotides. In some embodiments, the coding sequence comprises 0 to 10 CpG dinucleotides.

In some embodiments, the coding sequence comprises an increased number of CpG dinucleotides as compared to the wild type RPE65 nucleotide sequence. In some embodiments, the coding sequence comprises about 200% of CpG dinucleotides as compared to the wild type RPE65 nucleotide sequence. In some embodiments, the coding sequence comprises about 300% of CpG dinucleotides as compared to the wild type RPE65 nucleotide sequence. In some embodiments, the coding sequence comprises about 400% of CpG dinucleotides as compared to the wild type RPE65 nucleotide sequence. In some embodiments, the coding sequence comprises about 500% of CpG dinucleotides as compared to the wild type RPE65 nucleotide sequence. In some embodiments, the coding sequence comprises about 600% of CpG dinucleotides as compared to the wild type RPE65 nucleotide sequence. In some embodiments, the coding sequence comprises about 700% of CpG dinucleotides as compared to the wild type RPE65 nucleotide sequence.

In some embodiments, the coding sequence comprises no less than 50 CpG dinucleotides. In some embodiments, the coding sequence comprises no less than 100 CpG dinucleotides. In some embodiments, the coding sequence comprises no less than 150 CpG dinucleotides. In some embodiments, the coding sequence comprises no less than 200 CpG dinucleotides. In some embodiments, the coding sequence comprises no less than 250 CpG dinucleotides. In some embodiments, the coding sequence comprises no less than 300 CpG dinucleotides. In some embodiments, the coding sequence comprises about 50 to 300 CpG dinucleotides. In some embodiments, the coding sequence comprises about 100 to 250 CpG dinucleotides. In some embodiments, the coding sequence comprises about 150 to 200 CpG dinucleotides. In some embodiments, the coding sequence comprises about 150 CpG dinucleotides. In some embodiments, the coding sequence comprises about 100 CpG dinucleotides.

In some embodiments, the coding sequence is selected from the group consisting of

SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In some embodiments, the coding sequence is SEQ ID NO: 2. In some embodiments, the coding sequence has at least 80% identity to SEQ ID No: 2. In some embodiments, the coding sequence has at least 90% identity to SEQ ID No: 2. In some embodiments, the coding sequence has at least 95% identity to SEQ ID No: 2. In some embodiments, the coding sequence is SEQ ID NO: 3. In some embodiments, the coding sequence has at least 80% identity to SEQ ID No: 3. In some embodiments, the coding sequence has at least 90% identity to SEQ ID No: 3. In some embodiments, the coding sequence has at least 95% identity to SEQ ID No: 3. In some embodiments, the coding sequence is SEQ ID NO: 4. In some embodiments, the coding sequence has at least 80% identity to SEQ ID No: 4. In some embodiments, the coding sequence has at least 90% identity to SEQ ID No: 4. In some embodiments, the coding sequence has at least 95% identity to SEQ ID No: 4. In some embodiments, the coding sequence is SEQ ID NO: 5. In some embodiments, the coding sequence has at least 80% identity to SEQ ID No: 5. In some embodiments, the coding sequence has at least 90% identity to SEQ ID No: 5. In some embodiments, the coding sequence has at least 95% identity to SEQ ID No: 5. In some embodiments, the coding sequence is SEQ ID NO: 6. In some embodiments, the coding sequence has at least 80% identity to SEQ ID No: 6. In some embodiments, the coding sequence has at least 90% identity to SEQ ID No: 6. In some embodiments, the coding sequence has at least 95% identity to SEQ ID No: 6. In some embodiments, the coding sequence is SEQ ID NO: 7. In some embodiments, the coding sequence has at least 80% identity to SEQ ID No: 7. In some embodiments, the coding sequence has at least 90% identity to SEQ ID No: 7. In some embodiments, the coding sequence has at least 95% identity to SEQ ID No: 7. In some embodiments, the coding sequence is SEQ ID NO: 8. In some embodiments, the coding sequence has at least 80% identity to SEQ ID No: 8. In some embodiments, the coding sequence has at least 90% identity to SEQ ID No: 8. In some embodiments, the coding sequence has at least 95% identity to SEQ ID No: 8. In some embodiments, the coding sequence is SEQ ID NO: 9. In some embodiments, the coding sequence has at least 80% identity to SEQ ID No: 9. In some embodiments, the coding sequence has at least 90% identity to SEQ ID No: 9. In some embodiments, the coding sequence has at least 95% identity to SEQ ID No: 9. In some embodiments, the coding sequence is SEQ ID NO: 10. In some embodiments, the coding sequence has at least 80% identity to SEQ ID No: 10. In some embodiments, the coding sequence has at least 90% identity to SEQ ID No: 10. In some embodiments, the coding sequence has at least 95% identity to SEQ ID No: 10.

In some embodiments, the RPE65 polypeptide is expressed in a host cell after infection of the host cell by the rAAV particles. In some embodiments, the expression level of the RPE65 polypeptide of the rAAV particle in the host cell is higher than the expression level of the rAAV particle containing the wild type RPE65 coding sequence in the host cell. In some embodiments, the expression level of the RPE65 polypeptide of the rAAV particle in the host cell is approximately 1.1 times the expression level of the rAAV particle containing the wild type RPE65 coding sequence in the host cell. In some embodiments, the expression level of the RPE65 polypeptide of the rAAV particle in the host cell is approximately 1.2 times the expression level of the rAAV particle containing the wild type RPE65 coding sequence in the host cell. In some embodiments, the expression level of the RPE65 polypeptide of the rAAV particle in the host cell is approximately 1.3 times the expression level of the rAAV particle containing the wild type RPE65 coding sequence in the host cell. In some embodiments, the expression level of the RPE65 polypeptide of the rAAV particle in the host cell is approximately 1.4 times the expression level of the rAAV particle containing the wild type RPE65 coding sequence in the host cell. In some embodiments, the expression level of the RPE65 polypeptide of the rAAV particle in the host cell is approximately 1.5 times the expression level of the rAAV particle containing the wild type RPE65 coding sequence in the host cell. In some embodiments, the expression level of the RPE65 polypeptide of the rAAV particle in the host cell is approximately 2 times the expression level of the rAAV particle containing the wild type RPE65 coding sequence in the host cell. In some embodiments, the expression level of the RPE65 polypeptide of the rAAV particle in the host cell is approximately 2.5 times the expression level of the rAAV particle containing the wild type RPE65 coding sequence in the host cell. In some embodiments, the expression level of the RPE65 polypeptide of the rAAV particle in the host cell is approximately 3 times the expression level of the rAAV particle containing the wild type RPE65 coding sequence in the host cell. In some embodiments, the expression level of the RPE65 polypeptide of the rAAV particle in the host cell is approximately 3.5 times the expression level of the rAAV particle containing the wild type RPE65 coding sequence in the host cell. In some embodiments, the expression level of the RPE65 polypeptide of the rAAV particle in the host cell is approximately 4 times the expression level of the rAAV particle containing the wild type RPE65 coding sequence in the host cell. In some embodiments, the expression level of the RPE65 polypeptide of the rAAV particle in the host cell is approximately 4.5 times the expression level of the rAAV particle containing the wild type RPE65 coding sequence in the host cell. In some embodiments, the expression level of the RPE65 polypeptide of the rAAV particle in the host cell is approximately 5 times the expression level of the rAAV particle containing the wild type RPE65 coding sequence in the host cell. In some embodiments, the expression level of the RPE65 polypeptide of the rAAV particle in the host cell is approximately 5.5 times the expression level of the rAAV particle containing the wild type RPE65 coding sequence in the host cell. In some embodiments, the expression level of the RPE65 polypeptide of the rAAV particle in the host cell is approximately 6 times the expression level of the rAAV particle containing the wild type RPE65 coding sequence in the host cell. In some embodiments, the expression level of the RPE65 polypeptide of the rAAV particle in the host cell is approximately 6.5 times the expression level of the rAAV particle containing the wild type RPE65 coding sequence in the host cell. In some embodiments, the expression level of the RPE65 polypeptide of the rAAV particle in the host cell is approximately 7 times the expression level of the rAAV particle containing the wild type RPE65 coding sequence in the host cell. In some embodiments, the expression level of the RPE65 polypeptide of the rAAV particle in the host cell is approximately 7.5 times the expression level of the rAAV particle containing the wild type RPE65 coding sequence in the host cell. In some embodiments, the expression level of the RPE65 polypeptide of the rAAV particle in the host cell is approximately 8 times the expression level of the rAAV particle containing the wild type RPE65 coding sequence in the host cell. In some embodiments, the expression level of the RPE65 polypeptide of the rAAV particle in the host cell is approximately 8.5 times the expression level of the rAAV particle containing the wild type RPE65 coding sequence in the host cell. In some embodiments, the expression level of the RPE65 polypeptide of the rAAV particle in the host cell is approximately 9 times the expression level of the rAAV particle containing the wild type RPE65 coding sequence in the host cell. In some embodiments, the expression level of the RPE65 polypeptide of the rAAV particle in the host cell is approximately 9.5 times the expression level of the rAAV particle containing the wild type RPE65 coding sequence in the host cell. In some embodiments, the expression level of the RPE65 polypeptide of the rAAV particle in the host cell is approximately 10 times the expression level of the rAAV particle containing the wild type RPE65 coding sequence in the host cell. In some embodiments, the expression level of the RPE65 polypeptide of the rAAV particle in the host cell is approximately 11 times the expression level of the rAAV particle containing the wild type RPE65 coding sequence in the host cell. In some embodiments, the expression level of the RPE65 polypeptide of the rAAV particle in the host cell is approximately 12 times the expression level of the rAAV particle containing the wild type RPE65 coding sequence in the host cell. In some embodiments, the expression level of the RPE65 polypeptide of the rAAV particle in the host cell is approximately 13 times the expression level of the rAAV particle containing the wild type RPE65 coding sequence in the host cell. In some embodiments, the expression level of the RPE65 polypeptide of the rAAV particle in the host cell is approximately 14 times the expression level of the rAAV particle containing the wild type RPE65 coding sequence in the host cell. In some embodiments, the expression level of the RPE65 polypeptide of the rAAV particle in the host cell is approximately 15 times the expression level of the rAAV particle containing the wild type RPE65 coding sequence in the host cell. In some embodiments, the expression level of the RPE65 polypeptide of the rAAV particle in the host cell is approximately 20 times the expression level of the rAAV particle containing the wild type RPE65 coding sequence in the host cell. In some embodiments, the expression level of the RPE65 polypeptide of the rAAV particle in the host cell is approximately 25 times the expression level of the rAAV particle containing the wild type RPE65 coding sequence in the host cell. In some embodiments, the expression level of the RPE65 polypeptide of the rAAV particle in the host cell is approximately 30 times the expression level of the rAAV particle containing the wild type RPE65 coding sequence in the host cell. In some embodiments, the expression level of the RPE65 polypeptide of the rAAV particle in the host cell is approximately 35 times the expression level of the rAAV particle containing the wild type RPE65 coding sequence in the host cell. In some embodiments, the expression level of the RPE65 polypeptide of the rAAV particle in the host cell is approximately 40 times the expression level of the rAAV particle containing the wild type RPE65 coding sequence in the host cell. In some embodiments, the expression level of the RPE65 polypeptide of the rAAV particle in the host cell is approximately 45 times the expression level of the rAAV particle containing the wild type RPE65 coding sequence in the host cell. In some embodiments, the expression level of the RPE65 polypeptide of the rAAV particle in the host cell is approximately 50 times the expression level of the rAAV particle containing the wild type RPE65 coding sequence in the host cell.

In some embodiments, the stability of the RPE65 messenger ribonucleic acid (mRNA) expressed by the rAAV particles in the host cell is higher than that of the RPE65 mRNA expressed by the wild type RPE65 coding sequence. In some embodiments, the RPE65 mRNA expressed by the rAAV particles has a longer half-life in the host cell compared to the RPE65 mRNA expressed by the wild type RPE65 coding sequence. In some embodiments, the RPE65 mRNA expressed by the rAAV particles has a half-life increased by about 10% compared to the RPE65 mRNA expressed by the wild type RPE65 coding sequence. In some embodiments, the RPE65 mRNA expressed by the rAAV particles has a half-life increased by about 20% compared to the RPE65 mRNA expressed by the wild type RPE65 coding sequence. In some embodiments, the RPE65 mRNA expressed by the rAAV particles has a half-life increased by about 30% compared to the RPE65 mRNA expressed by the wild type RPE65 coding sequence. In some embodiments, the RPE65 mRNA expressed by the rAAV particles has a half-life increased by about 40% compared to the RPE65 mRNA expressed by the wild type RPE65 coding sequence. In some embodiments, the RPE65 mRNA expressed by the rAAV particles has a half-life increased by about 50% compared to the RPE65 mRNA expressed by the wild type RPE65 coding sequence. In some embodiments, the RPE65 mRNA expressed by the rAAV particles has a half-life increased by about 60% compared to the RPE65 mRNA expressed by the wild type RPE65 coding sequence. In some embodiments, the RPE65 mRNA expressed by the rAAV particles has a half-life increased by about 70% compared to the RPE65 mRNA expressed by the wild type RPE65 coding sequence. In some embodiments, the RPE65 mRNA expressed by the rAAV particles has a half-life increased by about 80% compared to the RPE65 mRNA expressed by the wild type RPE65 coding sequence. In some embodiments, the RPE65 mRNA expressed by the rAAV particles has a half-life increased by about 90% compared to the RPE65 mRNA expressed by the wild type RPE65 coding sequence. In some embodiments, the RPE65 mRNA expressed by the rAAV particles has a half-life increased by about 100% compared to the RPE65 mRNA expressed by the wild type RPE65 coding sequence.

In some embodiments, the stability of the RPE65 polypeptide expressed by the rAAV particles in the host cell is higher than that of the RPE65 polypeptide expressed by the wild type RPE65 coding sequence. In some embodiments, the RPE65 polypeptide expressed by the rAAV particles has a longer half-life in the host cell compared to the RPE65 polypeptide expressed by the wild type RPE65 coding sequence. In some embodiments, the RPE65 polypeptide expressed by the rAAV particles has a half-life increased by about 10% compared to the RPE65 polypeptide expressed by the wild type RPE65 coding sequence. In some embodiments, the RPE65 polypeptide expressed by the rAAV particles has a half-life increased by about 20% compared to the RPE65 polypeptide expressed by the wild type RPE65 coding sequence. In some embodiments, the RPE65 polypeptide expressed by the rAAV particles has a half-life increased by about 30% compared to the RPE65 polypeptide expressed by the wild type RPE65 coding sequence. In some embodiments, the RPE65 polypeptide expressed by the rAAV particles has a half-life increased by about 40% compared to the RPE65 polypeptide expressed by the wild type RPE65 coding sequence. In some embodiments, the RPE65 polypeptide expressed by the rAAV particles has a half-life increased by about 50% compared to the RPE65 polypeptide expressed by the wild type RPE65 coding sequence. In some embodiments, the RPE65 polypeptide expressed by the rAAV particles has a half-life increased by about 60% compared to the RPE65 polypeptide expressed by the wild type RPE65 coding sequence. In some embodiments, the RPE65 polypeptide expressed by the rAAV particles has a half-life increased by about 70% compared to the RPE65 polypeptide expressed by the wild type RPE65 coding sequence. In some embodiments, the RPE65 polypeptide expressed by the rAAV particles has a half-life increased by about 80% compared to the RPE65 polypeptide expressed by the wild type RPE65 coding sequence. In some embodiments, the RPE65 polypeptide expressed by the rAAV particles has a half-life increased by about 90% compared to the RPE65 polypeptide expressed by the wild type RPE65 coding sequence. In some embodiments, the RPE65 polypeptide expressed by the rAAV particles has a half-life increased by about 100% compared to the RPE65 polypeptide expressed by the wild type RPE65 coding sequence.

In some embodiments, the RPE65 DNA contained in the rAAV particles has lower immunogenicity in the subject than the wild type RPE65 DNA. In some embodiments, the RPE65 mRNA expressed by the rAAV particles has lower immunogenicity in the subject than the RPE65 mRNA expressed by the wild type RPE65 coding sequence.

In some embodiments, the rAAV particle further comprises an AAV protein. In some embodiments, the AAV protein may be from any AAV serotype. In some embodiments, the AAV protein may be from AAV serotype 1 (AAV1), AAV serotype 2 (AAV2), AAV serotype 2 variants (such as AAV2.7m8, AAV2(quad Y-F), and AAV2tYF), AAV serotype 3 (AAV3, including serotypes 3A and 3B), AAV serotype 4 (AAV4) , AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 12 (AAV12), AAV serotype 13 (AAV13), AAV-Rh10, AAV-Rh74, AAV-2i8 and any other AAVs known. In some embodiments, the AAV protein has at least 75%, 80%, 85%, 90%, 95% or more identity to the wild type AAV proteins derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the AAV protein has one or more amino acid substitutions, deletions and/or additions compared to the wild type AAV proteins derived from AAV1, AAV2, AAV2 variants (such as AAV2.7m8, AAV2(quad Y-F), and AAV2tYF), AAV3 (including AAV3A and 3B), AAV4, AAV5, AAV6, AAV7, AAV8, AAV9, AAV10, AAV11, AAV12, AAV13, AAV-Rh10, AAV-Rh74 or AAV-2i8. In some embodiments, the AAV protein is from serotype AAV2 or variants thereof, serotype AAV5 or variants thereof, or serotype AAV8 or variants thereof.

In some embodiments, the nucleotide sequence further comprises a promoter, and the promoter is operably linked to the coding sequence. In some embodiments, the promoter is CMV, CAG, MNDU3, PGK, EF1a, Ubc promoter or ocular tissue specific promoter. In some embodiments, the ocular tissue-specific promoter is selected from the RPE 65 gene promoter, human retinal binding protein (CRALBP) gene promoter, murine 11-cis-retinol dehydrogenase (RDH) gene promoter, rhodopsin promoter, rhodopsin kinase promoter, tissue inhibitor of metalloproteinase 3 (Timp3) promoter, photoreceptor retinol binding protein promoter and vitelliform macular dystrophy 2 promoter, or interphotoreceptor retinoid-binding protein (IRBP) promoters.

In some embodiments, the expression cassette polynucleotide sequence further comprises a WPRE sequence at the 3′ end.

In some embodiments, the expression cassette polynucleotide sequence further comprises a polyadenylation sequence or a “poly(A) sequence” at the 3′ end. In some embodiments, the length of the polyadenylation sequence or “poly(A) sequence” may range from about 1-500 bp. In some embodiments, the length of the polyadenylation sequence or “poly(A) sequence” may be, but is not limited to, 1, 2, 3, 4, 5, 6, 7, 8, 9, 10, 20, 30, 50, 100, 200, or 500 nucleotides. In some embodiments, the poly(A) sequence is one of SV40pA, hGHpA and bGHpA.

In some embodiments, the polynucleotide further comprises other regulatory sequences. The regulatory sequences include, but are not limited to, inverted terminal repeats (ITR), enhancers, splicing signals, polyadenylation signals, stuffer sequences, terminators, protein degradation signals, internal ribosome entry elements (IRES), 2A sequences, and the like.

In some embodiments, the polynucleotide further comprises an enhancer region. In some embodiments, the enhancer region comprises an SV40 enhancer, an immediate-early cytomegalovirus enhancer, an IRBP enhancer, and an enhancer derived from an immunoglobulin gene. In some embodiments, the enhancer region is located upstream of the CMV, CAG, MNDU3, PGK, and EF1a promoter. In some embodiments, the enhancer is located upstream of the ocular tissue-specific promoter. In some embodiments, the enhancer region is located downstream of the CMV, CAG, MNDU3, PGK, and EF1a promoter. In some embodiments, the enhancer is located downstream of the ocular tissue-specific promoter.

In some embodiments, the polynucleotide further comprises an inverted terminal repeat (ITR) sequence. In some embodiments, the polynucleotide comprises at least one inverted terminal repeat (ITR) sequence. In some embodiments, the polynucleotide comprises two inverted terminal repeat sequences (ITRs). In some embodiments, the two ITRs are the same. In some embodiments, the two ITRs are different from each other. In some embodiments, the inverted terminal repeat sequences (ITRs) are ITRs derived from AAV. In some embodiments, the ITR may be derived from ITRs of AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known. In some embodiments, the ITR has one or more base mutations, insertions or deletions compared to wild type ITRs derived from AAV1, AAV2, AAV3 (including AAV3A and 3B), AAV4, AAV5, AAV6, AAV7, AAV8, AAV9, AAV10, AAV11, AAV12, AAV13, AAV-Rh10, AAV-Rh74, AAV-2i8 and any other AAVs known, as long as it retains the desired function as a terminal repeat sequence, such as replication of the target gene, packaging and/or integration of virus particles, and the like.

In some embodiments, the polynucleotide further comprises one or more stuffer sequences. In some embodiments, the stuffer sequence is located upstream of the CMV, CAG, MNDU3, PGK, and EF1a promoter sequence. In some embodiments, the stuffer sequence is located downstream of the CMV, CAG, MNDU3, PGK, and EF1a promoter sequence. In some embodiments, the stuffer sequence is located upstream of the ocular tissue-specific promoter. In some embodiments, the stuffer sequence is located downstream of the ocular tissue-specific promoter. In some embodiments, the stuffer sequence is located at the 5′ end of the 5′ ITR sequence. In some embodiments, the stuffer sequence is located at the 3′ end of the 5′ ITR sequence. In some embodiments, the stuffer sequence is located at the 5′ end of the 3′ ITR sequence. In some embodiments, the stuffer sequence is located at the 3′ end of the 3′ ITR sequence.

In some embodiments, the length of the stuffer sequence may be about 0.1 kb-5 kb, such as, but are not limited to, 0.1 kb, 0.2 kb, 0.3 kb, 0.4 kb, 0.5 kb, 0.6 kb, 0.7 kb, 0.8 kb, 0.9 kb, 1 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb or 5.0 kb.

In some embodiments, the polynucleotide further comprises sequences encoding one other therapeutic protein. In some embodiments, the therapeutic protein is selected from the group consisting of: ATP-binding cassette sub-family A member 4 (ABCA4), retinol dehydrogenase 12 (RDH12), retinol dehydrogenase 8 (RDH8), retinol binding protein 3 (RBP 3), retinol binding protein 1 (RBP 1), lecithin retinol acyltransferase (LRAT), retinaldehyde binding protein 1 (Rlbp1), retinol dehydrogenase 10 (RDH10), and retinol dehydrogenase hydrogenase 11 (RDH11).

In some embodiments, the sequences encoding the other therapeutic proteins are linked to the coding sequence by a sequence encoding a linker. In some embodiments, the linker is a cleavable linker. In some embodiments, the cleavable linker comprises a sequence of a 2A peptide. In some embodiments, the 2A peptide may be selected from 2A peptides derived from aphthoviruses or cardioviruses, such as 2A peptides derived from foot-and-mouth disease virus (FMDV), equine rhinitis A virus (ERAV), Thoseaasigna Virus (TaV) or porcine teschen virus (PTV-1).

In some embodiments, the polynucleotide is sequence-optimized. In some embodiments, the promoter is optimized. In some embodiments, the stuffer sequence is optimized. In some embodiments, the other therapeutic proteins are optimized. In some embodiments, the linker sequence is optimized.

In some embodiments, the polynucleotide comprises no more than 500 CpG dinucleotides. In some embodiments, the polynucleotide comprises no more than 450 CpG dinucleotides. In some embodiments, the polynucleotide comprises no more than 400 CpG dinucleotides. In some embodiments, the polynucleotide comprises no more than 350 CpG dinucleotides. In some embodiments, the polynucleotide comprises no more than 300 CpG dinucleotides. In some embodiments, the polynucleotide comprises no more than 250 CpG dinucleotides. In some embodiments, the polynucleotide comprises no more than 200 CpG dinucleotides.

In some embodiments, the polynucleotide comprises about 200 to 500 CpG dinucleotides. In some embodiments, the polynucleotide comprises about 250 to 450 CpG dinucleotides. In some embodiments, the polynucleotide comprises about 300 to 400 CpG dinucleotides. In some embodiments, the polynucleotide comprises about 200 to 400 CpG dinucleotides. In some embodiments, the polynucleotide comprises about 200 to 300 CpG dinucleotides. In some embodiments, the polynucleotide comprises about 210 to 290 CpG dinucleotides. In some embodiments, the polynucleotide comprises about 220 to 280 CpG dinucleotides. In some embodiments, the polynucleotide comprises about 230 to 270 CpG dinucleotides. In some embodiments, the polynucleotide comprises about 240 to 260 CpG dinucleotides. In some embodiments, the polynucleotide comprises about 250 CpG dinucleotides. In another aspect, the present disclosure provides a method for preparing the recombinant adeno-associated virus (rAAV) particle, comprising introducing the herein described expression cassette polynucleotide sequence in a host cell. In another aspect, the present disclosure provides a recombinant adeno-associated virus (rAAV) particle, which is prepared by a method that comprises introducing the herein described expression cassette polynucleotide sequence in a host cell. In some embodiments, the method comprises expressing the herein described expression cassette polynucleotide sequence in the host cell. In some embodiments, the host cell is a human cell, animal cell, or insect cell. In some embodiments, the host cell is a human cell. In some embodiments, the host cell is the Sf9 cell. In some embodiments, the host cell is the HEK293 cell or a derivative thereof. In some embodiments, the host cell is the HEK293T cell. In some embodiments, the host cell is the HEK293FT cell. In some embodiments, the host cell is an insect cell. In some embodiments, the method comprises generating bacmid DNA and/or baculovirus. In some embodiments, the method comprises generating RPE65 expression sequence bacmid DNA. In some embodiments, the method comprises generating rAAV cap expression sequence bacmid DNA. In some embodiments, the method comprises transfecting a host cell with the bacmid DNA to produce baculoviruses. In some embodiments, the method comprises transfecting a host cell with the RPE65 expression sequence bacmid DNA to produce baculoviruses. In some embodiments, the method comprises transfecting a host cell with the rAAV cap expression sequence bacmid DNA to produce baculoviruses. In some embodiments, the method further comprises mixing the two baculoviruses to infect a host cell (such as Sf9 cell) to obtain packaged rAAV/RPE65-optimized virus particles of the present disclosure.

In some embodiments, the composition of the present disclosure can be delivered into the host cell by any method known in the art. In some embodiments, the method includes, but is not limited to, electroporation, calcium phosphate precipitation, liposome mediation, and the like. In some embodiments, the composition is stably transfected into the host cell. In some embodiments, the composition is transiently transfected into the host cell. In some embodiments, the host cell is used to produce the rAAV virus particles.

If necessary, the rAAV virus particles can be isolated and purified from the host cell according to conventional methods known to those skilled in the art. For example, the rAAV virus particles can be purified using centrifugation, HPLC, hydrophobic interaction chromatography (HIC), anion exchange chromatography, cation exchange chromatography, size exclusion chromatography, ultrafiltration, gel electrophoresis, affinity chromatography, and/or other purification techniques.

### Pharmaceutical Composition

In one aspect, provided herein is a pharmaceutical composition comprising the described rAAV particle or the described composition. In some embodiments, the pharmaceutical composition comprises the rAAV particles of the present disclosure and a pharmaceutically acceptable carrier or excipient.

As used herein, “pharmaceutically or therapeutically acceptable carrier or excipient” refers to a carrier medium that does not interfere with the effectiveness of the biological activity of the active ingredient and is non-toxic to the host or patient. The type of carrier used in the pharmaceutical formulation will depend on the method of administration of the therapeutic compound. Many methods of preparing pharmaceutical compositions for multiple routes of administration are well known in the art. “Pharmaceutically acceptable ophthalmic carrier” refers to a pharmaceutically acceptable carrier or excipient that can be used to directly or indirectly deliver the rAAV virus particles of the present disclosure to, on or near the eye.

In some embodiments of the disclosure, the pharmaceutical composition is prepared by dissolving the rAAV virus particles of the present disclosure in a suitable solvent. Suitable solvents include, but are not limited to, water, saline solutions (e.g., NaCl), buffer solutions (e.g., phosphate-buffered saline (PBS)), or other solvents. In certain embodiments, the viral particle pharmaceutical composition may include a surfactant (e.g., Poloxamer, pluronic acid F68). In certain embodiments, the solvent is sterile. In certain embodiments, the viral particle pharmaceutical composition comprises sodium chloride, sodium phosphate and poloxamer. In some embodiments, the pharmaceutical composition does not comprise any preservatives.

In some embodiments, the pharmaceutical composition is a suspension. In some embodiments, the pharmaceutical composition is a solution.

A pharmaceutical composition described herein can comprise any suitable amount of rAAV particles. In some embodiments, the pharmaceutical composition comprises 1×10⁹ to 1×10¹⁴ vector genomes (vg) per mL. In some embodiments, the pharmaceutical composition comprises 1×10¹⁰ to 1×10¹³ vg per mL. In some embodiments, the pharmaceutical composition comprises 5×10¹⁰ to 5×10¹² vg per mL. In some embodiments, the pharmaceutical composition comprises 1×10¹¹to 1×10¹² vg per mL. In some embodiments, the pharmaceutical composition comprises 0.1 to 5 mL in volume. In some embodiments, the pharmaceutical composition comprises 0.2 to 0.5 mL in volume. In some embodiments, the pharmaceutical composition comprises 0.1 to 1 mL in volume.

### Treatment Method

In one aspect, the present application provides a method for treating an inherited retinal disease, such as one caused by mutations of one or both copies of RPE65 gene. In one aspect, the present application provides a method for treating Leber congenital amaurosis (LCA). In some embodiments, the method comprises administering a therapeutically effective amount of the rAAV virus particles described herein and/or the pharmaceutical composition of the present disclosure to a subject in need thereof. In some embodiments, the subject has an inherited retinal disease caused by mutations of both copies of RPE65 gene. In some embodiments, the subject has LCA.

In some embodiments, the rAAV virus particles and/or the pharmaceutical composition can be administered to the subject by any suitable method known in the art. In some embodiments, the rAAV virus particles and/or the pharmaceutical composition may be administered locally to the eye, such as by subconjunctival, retrobulbar, periocular, intravitreal, subretinal, suprachoroidal, or intraocular administration. In some embodiments, the rAAV virus particles and/or the pharmaceutical composition is administered via subretinal injection.

In some embodiments, the pharmaceutical composition comprising the rAAV viral particles is provided in a therapeutically effective amount that achieves the desired biological effect at a medically acceptable level of toxicity. The dosage can vary according to the route of administration and the severity of the disease. The dosage can also be adjusted according to the weight, age, gender and/or degree of symptoms of each patient to be treated. The precise dosage and route of administration will ultimately be determined by the attending doctor or veterinarian. Understandably, routine dosage changes may be required depending on the age and weight of the patient and the severity of the condition to be treated.

In some embodiments, the therapeutically effective amount is generally about 1×10⁵ to 1×10¹³ rAAV virus particles. In some embodiments, the therapeutically effective amount is 1×10⁶ to 1×10¹³ rAAV virus particles. In some embodiments, the therapeutically effective amount is 1×10⁷ to 1×10¹³ rAAV virus particles. In some embodiments, the therapeutically effective amount is 1×10⁸ to 1×10¹³ rAAV virus particles. In some embodiments, the therapeutically effective amount is 1×10⁹ to 1×10¹³ rAAV virus particles. In some embodiments, the therapeutically effective amount is 1×10¹⁰ to 1×10¹³ rAAV virus particles. In some embodiments, the therapeutically effective amount is 1×10¹¹ to 1×10¹³ rAAV virus particles. In some embodiments, the therapeutically effective amount is 1×10¹² to 1×10¹³ rAAV virus particles. In some embodiments, the therapeutically effective amount is generally about 1×10⁶ to 1×10¹² rAAV virus particles. In some embodiments, the therapeutically effective amount is generally about 1×10⁷ to 1×10¹² rAAV virus particles. In some embodiments, the therapeutically effective amount is generally about 1×10⁸ to 1×10¹² rAAV virus particles. In some embodiments, the therapeutically effective amount is generally about 1×10⁹ to 1×10¹² rAAV virus particles. In some embodiments, the therapeutically effective amount is generally about 1×10¹⁰ to 1×10¹² rAAV virus particles. In some embodiments, the therapeutically effective amount is 1×10⁹ to 1×10¹⁰ rAAV virus particles.

In some embodiments, the therapeutically effective amount is about 1×10⁵ to 1×10²⁰ vector genomes (vg) per dose. In some embodiments, the therapeutically effective amount is 1×10⁶ to 1×10¹⁶ vg per dose. In some embodiments, the therapeutically effective amount is 1×10⁷ to 1×10¹⁴ vg per dose. In some embodiments, the therapeutically effective amount is 1×10⁸ to 1×10¹³ vg per dose. In some embodiments, the therapeutically effective amount is 1×10⁹ to 1×10¹³ vg per dose. In some embodiments, the therapeutically effective amount is 1×10¹⁰ to 1×10¹³ vg per dose. In some embodiments, the therapeutically effective amount is 1×10¹¹ to 1×10¹⁰ vg per dose. In some embodiments, the therapeutically effective amount is 1×10¹² to 1×10¹⁰ vg per dose. In some embodiments, the therapeutically effective amount is generally about 1×10⁶ to 1×10¹² vg per dose. In some embodiments, the therapeutically effective amount is generally about 1×10⁷ to 1×10¹² vg per dose. In some embodiments, the therapeutically effective amount is generally about 1×10⁸ to 1×10¹² vg per dose. In some embodiments, the therapeutically effective amount is generally about 1×10⁹ to 1×10¹² vg per dose. In some embodiments, the therapeutically effective amount is generally about 1×10¹⁰ to 1×10¹² vg per dose. In some embodiments, the therapeutically effective amount is 1×10⁹ to 1×10¹⁰ vg per dose.

In some embodiments, the delivered volume is about 0.01 mL-1 mL. In some embodiments, the delivered volume is about 0.05 mL-1 mL. In some embodiments, the delivered volume is about 0.1 mL-1 mL. In some embodiments, the delivered volume is about 0.5 mL-1 mL. In some embodiments, the delivered volume is about 0.1 mL-0.5 mL. In some embodiments, the delivered volume is about 0.01 mL-0.5 mL. In some embodiments, the delivered volume is about 0.05 mL-0.5 mL. In some embodiments, the delivered volume is about 0.05 mL-1 mL.

In some embodiments, the frequency of administration may be at least once per day, including 2, 3, 4, or 5 times per day. In some embodiments, the treatment may last for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 60 days, 70 days, 80 days, 90 days, 100 days, 150 days, 200 days, 250 days, 300 days, 400 days, 500 days, 750 days, 1000 days or more than 1000 days.

In some embodiments, the administration comprises diluting the pharmaceutical composition. For example, the pharmaceutical composition can be diluted from 1:1 to 1: 100 ratio prior to administration. In some embodiments, the pharmaceutical composition is diluted 1:10 prior to administration.

In some embodiments, the administration comprises a single dose per eye. The administration to each eye of the subject can be one the same or different days. In some embodiments, the administration to each eye of the subject are performed on separate days, e.g., at least 1 day, 2 days, 3 days, 4 days, 5 days, 6 days, 7 days, or 10 days apart. In some embodiments, the administration to each eye of the subject are performed at most 45 days, 30 days, 20 days, 15 day, 10 day, 7 days, or 3 days apart. In some embodiments, the administration to each eye of the subject are performed no fewer than 6 days apart.

In some embodiments, a second therapeutic agent can be administered concurrently or sequentially with the described pharmaceutical composition. In some embodiments, the second therapeutic agent is systemic oral corticosteroids. For example, the oral corticosteroid can be administered at 0.1 to 40 mg/kg/day for a total of 1 to 30 days. In some embodiments, the oral corticosteroid is administered at 1 mg/kg/day for a total of 7 days. In some embodiments, the oral corticosteroid is administered starting 1, 2, 3, 4, 5, 6, or 7 days before the administration of the pharmaceutical composition. In some embodiments, the oral corticosteroid is administered with a tapering dose during the next 5, 6, 7, 8, 9, 10, 11, 12, 15 or more days after the administration of the pharmaceutical composition.

In some embodiments, the subject is at least 12 months of age. In some embodiments, the subject is an adult. In some embodiments, the subject is a child. In some embodiments, the subject is an elderly. In some embodiments, the subject is 1 to 18 year of age. In some embodiments, the subject is 4 to 12 year of age. In some embodiments, the subject is at least 18 years old.

### Kit

In another aspect, the present disclosure provides a kit for treating LCA, comprising the pharmaceutical composition of the present disclosure and instructions. In some embodiments, the instructions are used to indicate a method of administering the pharmaceutical composition to treat LCA.

In some embodiments, the kit further comprises a container. In some embodiments, the container is configured to deliver the pharmaceutical composition described herein. In some embodiments, the container comprises vials, droppers, bottles, tubes, and syringes. In some embodiments, the container is a dropper used to administer the pharmaceutical composition. In some embodiments, the container is a syringe used to administer the pharmaceutical composition.

Some embodiments of the present disclosure are further illustrated by the following examples, which should not be construed as limiting. Those skilled in the art will understand that the techniques disclosed in the following examples represent well-operated techniques in the practice of the embodiments of the disclosure described herein and, therefore, may be considered to constitute a preferred means for implementing these embodiments. However, it will be understood by those skilled in the art in light of this disclosure that many changes may be made in the specific embodiments disclosed herein without departing from the spirit and scope of the disclosure and still achieve the same or similar result.

### EXAMPLES

The following examples further illustrate the present disclosure. These examples are only intended to illustrate the present disclosure, and should not be construed as limiting the present disclosure.

### Example 1 Design and Cloning of Recombinant AAV Vector

The cap and rep coding sequences derived from rAAV together with their corresponding promoters were cloned into a pFastBacl vector, respectively, to obtain polynucleotides encoding the AAV proteins. The coding sequence of the capsid protein VP1 of rAAV is SEQ ID NO: 17; the coding sequence of the capsid protein VP2 is SEQ ID NO: 18; and the coding sequence of the capsid protein VP3 is SEQ ID NO: 19. The codons of the wild type nucleotide sequence encoding the RPE65 polypeptide shown in SEQ ID No: 1 were optimized. Specifically, the less frequently used codons in the RPE65 gene were synonymously replaced, while ensuring that the optimized nucleotide sequence of RPE65 contains an altered number of CpG dinucleotides. The amino acid sequence encoded by the codon-optimized RPE65 nucleotide sequences is consistent with the amino acid sequence of the RPE65 polypeptide shown in SEQ ID No: 1. In other words, the amino acid sequence encoded by the codon-optimized RPE65 nucleotide sequences is SEQ ID NO: 11. The optimized RPE65 nucleotide sequences are SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and SEQ ID NO: 10.

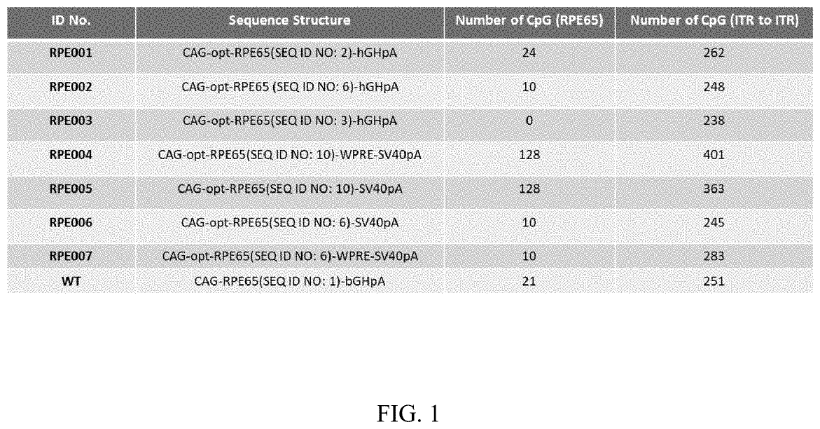

The optimized RPE65 nucleotide sequence of the present application, together with the CAG promoter (e.g. SEQ ID NO: 12) and the ITR sequences at both ends were cloned into a pFastBacl vector to obtain a polynucleotide containing the optimized RPE65 sequence. Wherein, as shown in FIG. 1, the 3′ end of the optimized RPE65 nucleotide sequence may be further followed by a poly(A) sequence hGHpA (e.g. SEQ ID NO: 13), SV40pA (e.g. SEQ ID NO: 15), or bGHpA (e.g. SEQ ID NO: 14). The 3′ end of the optimized RPE65 nucleotide sequence may be further followed by a WPRE sequence (e.g. SEQ ID NO: 16). The ITR sequences at both ends are ahead of the promoter and behind the poly(A).

### Example 2 Preparation Of Recombinant AAV Virus Particles

The polynucleotide encoding the AAV protein and the polynucleotide containing the optimized RPE65 sequence obtained in Example 1 were transformed into DH10Bac to produce Rep-Cap and RPE65 expression sequence bacmid DNA, respectively, and then separately transfected Sf9 insect cells to produce baculoviruses, followed by mixing the two baculoviruses to infect Sf9 cells to obtain packaged rAAV/RPE65-optimized virus particles of the present application. In addition, the polynucleotide encoding the AAV protein and the polynucleotide containing the optimized RPE65 sequence can also be co-transfected into HEK293 cells with the Helper plasmid vector to obtain the packaged rAAV/RPE65-optimized virus particles of the present application. Finally, the rAAV/RPE65-optimized virus particles were purified by gradient ultracentrifugation.

### Example 3 Expression of RPE65 in Host Cells In Vitro

Host cells transfected with RPE65-optimized expression plasmid can efficiently express RPE65 polypeptides. Compared with wild type RPE65 expression plasmid, the RPE65-optimized plasmid of the present application have a significantly higher expression efficiency of RPE65 polypeptides. After transfecting HEK293 cells with the RPE65-optimized polynucleotide of the present disclosure or the wild type RPE65 polynucleotide of the control plasmid, respectively, the HEK293 cells were collected and the expression levels of RPE65 were measured using Western blotting. The results showed that, taking the expression levels of wild type RPE65 in the control as a reference, the normalized expression levels of the other groups were as below (measured in 3 independent experiments): the expression levels of RPE001 were 0.36, 0.58 and 0.20; the expression levels of RPE002 were 0.76, 0.93 and 0.61; the expression levels of RPE003 were 2.43, 2.42 and 1.05; the expression levels of RPE004 were 8.00, 2.60 and 3.70; the expression levels of RPE005 were 5.48, 1.19 and 3.52; and the expression levels of RPE006 were 2.60, 1.31 and 3.02.

After infecting HEK293 cells with rAAV particles containing optimized RPE65 of the present disclosure and the wild type RPE65 of the control at a dose of MOI=1E5, positive cells expressing RPE65 proteins were measured using flow cytometry. The results were the following (measured in 2-3 independent experiments): the RPE65 positive cell rates of wild type AAV particles are 9.84% and 4.5%, the RPE65 positive cell rates of RPE001 were 4.71%, 3.5% and 5.02%; the RPE65 positive cell rates of RPE002 were 3.95%, 6.08%, and 4.74%; the RPE65 positive cell rates of RPE003 were 3.04%, 5.22% and 4.36%; the RPE65 positive cell rates of RPE004 were 28.8%, 31% and 27.4%; the RPE65 positive cell rates of RPE005 were 6.66% , 9.65% and 11%; the RPE65 positive cell rates of RPE006 were 9.25%, 13.6% and 14.1%; and the RPE65 positive cell rates of RPE007 were 18.9%, 27% and 22.6%.

After infecting HEK293 cells with rAAV particles containing optimized RPE65 of the present disclosure and the wild type RPE65 of the control at a dose of M01=5E5, positive cells expressing RPE65 proteins were measured using flow cytometry. The results were the following (measured in 2-3 independent experiments): the RPE65 positive cell rates of wild type AAV particles were 13.3% and 9.27%, the RPE65 positive cell rates of RPE001 were 7.88%, 9.11% and 2.94%; the RPE65 positive cell rates of RPE002 were 9.61%, 9.13% and 6.84%; the RPE65 positive cell rates of RPE003 were 7.73%, 11.5% and 7.39%; the RPE65 positive cell rates of RPE004 were 34.7%, 41% and 34.3%; the RPE65 positive cell rates of RPE005 were 22.9%, 20.9% and 12.4%; the RPE65 positive cell rates of RPE006 were 19.4%, 24.2% and 15.5%; and the RPE65 positive cell rates of RPE007 were 38.8%, 32.7% and 25.5%.

After infecting HEK293 cells with AAV particles containing optimized RPE65 of the present disclosure and the wild type RPE65 of the control at a dose of MOI=1E5, the expression levels were measured using Western blotting. The results showed that, taking the expression levels of the RPE65 protein in HEK293T cells infected with RPE001 AAV particles as a reference, the normalized expression levels of the other groups were as below (measured in 2-3 independent experiments): the expression levels of the wild type were 1.46 and 2.20; the expression levels of RPE002 were 1.17, 0.63 and 0.91; the expression levels of RPE003 were 1.61, 1.34 and 1.34; the expression levels of RPE004 were 11.63, 5.13 and 7.47; the expression levels of RPE005 were 2.60, 2.03 and 2.09; the expression levels of RPE006 were 2.70, 2.94 and 3.10; and the expression levels of RPE007 were 4.70, 8.24 and 7.20.

After infecting HEK293 cells with AAV particles containing optimized RPE65 of the present disclosure and the wild type RPE65 of the control at a dose of MOI=5E5, the expression levels of RPE65 were measured using Western blotting. The results showed that, taking the expression levels of the RPE65 protein in HEK293T cells infected with RPE001 AAV particles as a reference, the normalized expression levels of the other groups were as below (measured in 2-3 independent experiments): the expression levels of the wild type were 2.93 and 3.18; the expression levels of RPE002 were 0.91, 0.68 and 1.45; the expression levels of RPE003 were 2.10, 1.75 and 2.19; the expression levels of RPE004 were 17.44, 3.94 and 11.56; the expression levels of RPE005 were 3.95, 1.65 and 0.64; the expression levels of RPE006 were 3.73, 2.94 and 0.71; and the expression levels of RPE007 were 8.35, 4.51 and 10.15. The above results showed that both the infection rate and the expression level of RPE65 in the RPE65 optimized rAAV particles of the present disclosure were significantly higher than those of the wild type RPE65 AAV particles of the control.

### Example 4 Therapeutic Effect Of RAAV/Optimized RPE65 in B6(A)-RPE65^{RD12} Mice

B6(A)-Rpe65rd12 mice were used to determine the in vivo therapeutic effect of rAAV/optimized RPE65. Among them, the control used a blank vehicle buffer without rAAV, and the experimental group used the purified rAAV/RPE65-optimized virus particles, RPE003, RPE004, RPE006, RPE007, and WT generated from Example 2 for subretinal injection. Specifically, subretinal injections were performed on Rpe65rd12 mice 14 days after birth. A surgical microscope was used throughout the procedure, and the needle was inserted tangentially through the sclera, creating a wound having a self-sealing scleral tunnel. About 1 μl of the virus suspension was injected into the subretinal space, and both resulted in bullous retinal detachment visible by ophthalmoscope examination. One or both eyes of the mice were injected with a blank vehicle buffer without rAAV, or rAAV/RPE65-optimized virus particles. The dose of virus particles used for injection was 5×10⁹ vg for each eye.

After the injection, an electroretinogram (ERG) well known to those skilled in the art was used to observe the eyes of the mice. ERG is a non-invasive tool to test retinal function by measuring the electrical response of retinal cells to light stimulation. The ERG test is often used to assess ocular diseases and retinal degeneration, and it can be used in human or mouse eyes. There are two types of ERG tests: scotopic ERGs and photopic ERGs. Among them, the scotopic ERGs include a scotopic A-wave and scotopic B-wave. Scotopic ERGs use a low-intensity flash to induce the activation of rod cells after an overnight dark-adaptation to achieve maximal rod activiation and sensitivity. A-wave measures the function of the rod photoreceptor, and B-wave measures the retinal cell's downstream response to the stimulation of photoreceptors. The decrease and increase in amplitudes readout for either wave can indicate the disease progression and restoration of retinal function, respectively. Photopic ERGs use a high-intensity flash to induce the activation of cone cells and inhibit the response of rod cells after a period of light stimulation.

The use of ERG to assess the recovery of retinal function is a technical means well known to those skilled in the art. See, for example, an ERG protocol, including the details for experiment set-up, study materials, mouse preparation, ERG settings, and data processing, is described in Assessment of Murine Retinal Function by Electroretinography (G. Benchorin et al., Bio Protoc. 2017).

All mice were dark-adapted for at least 12 hours overnight before the day of experiment and kept dark-adapted by only using redfiltered light sources during the preparation. The mice were placed on the platform heated to 37° C. and were treated with eye drops containing atropine sulfate, phenylephrine hydrochloride, and proparacaine hydrochloride. The eye drops were then removed, and their eyes were then kept hydrated with an ointment. For both A-wave and B-wave are, the pulse intensity is 1 cd sec/m2. Microsoft Excel and GraphPad Prism are used to analyze the data.

One month after the injection treatment, the recovery of retinal function was evaluated by Scotopic ERG A-wave and B-wave. Scotopic ERGs were performed every month thereafter until 3 months after the injection (the last time point of the evaluation). The results are summarized in Tables 3 and 4.

TABLE 3
Scotopic ERG A-Wave Results

| | Mean | |
|---|---|---|
| Treatment Group | Amplitude(uV)* | n |
| Control - 1-month post-injection | B | 17 |
| Control - 2-month post-injection | B | 6 |
| Control - 3-month post-injection | B | 6 |
| RPE003 - 1-month post-injection | A | 9 |
| RPE003 - 2-month post-injection | A | 9 |
| RPE003 - 3-month post-injection | A | 5 |
| RPE004 - 1-month post-injection | B | 9 |
| RPE004 - 2-month post-injection | B | 9 |
| RPE004 - 3-month post-injection | B | 9 |
| RPE006 - 1-month post-injection | A | 11 |
| RPE006 - 2-month post-injection | A | 11 |
| RPE006 - 3-month post-injection | A | 11 |
| WT - 1-month post-injection | B | 12 |
| WT - 2-month post-injection | B | 6 |
| WT - 3-month post-injection | B | 6 |
| RPE007 - 1-month post-injection | A | 8 |
| RPE007 - 2-month post-injection | A | 8 |
| RPE007 - 3-month post-injection | A | 7 |

*10 uV ≤ A < 50 uV; and 0 < B < 10 uV.

TABLE 4
Scotopic ERG B-Wave Results

| | Mean | |
|---|---|---|
| Treatment Group | Amplitude(uV) * | n |
| Control - 1-month post-injection | C | 17 |
| Control - 2-month post-injection | C | 6 |
| Control - 3-month post-injection | C | 6 |
| RPE003 - 1-month post-injection | A | 9 |
| RPE003 - 2-month post-injection | A | 9 |
| RPE003 - 3-month post-injection | A | 5 |
| RPE004 - 1-month post-injection | B | 9 |
| RPE004 - 2-month post-injection | C | 9 |
| RPE004 - 3-month post-injection | C | 9 |
| RPE006 - 1-month post-injection | A | 11 |
| RPE006 - 2-month post-injection | A | 11 |
| RPE006 - 3-month post-injection | A | 11 |
| WT - 1-month post-injection | B | 12 |
| WT - 2-month post-injection | B | 6 |
| WT - 3-month post-injection | B | 6 |
| RPE007 - 1-month post-injection | A | 8 |
| RPE007 - 2-month post-injection | A | 8 |
| RPE007 - 3-month post-injection | A | 8 |

* 30 uV ≤ A < 100 uV; 15 uV < B < 30 uV; and 0 < C ≤ 15 uV.

In addition, this study also used retinal fundus imaging and optical coherence tomography (OCT) technology to evaluate the changes in retinal structure in the control and experimental groups. At the end of the study, samples of mouse eye tissues were collected for immunofluorescence staining and immunohistochemistry to evaluate the structure of the retina and the expression level of human RPE65 protein.

SEQUENCE LISTING
SEQ ID NO: 1
ATGTCTATCCAGGTTGAGCATCCTGCTGGTGGTTACAAGAAACTGTTTGAAACTGTG
GAGGAACTGTCCTCGCCGCTCACAGCTCATGTAACAGGCAGGATCCCCCTCTGGCT
CACCGGCAGTCTCCTTCGATGTGGGCCAGGACTCTTTGAAGTTGGATCTGAGCCATT
TTACCACCTGTTTGATGGGCAAGCCCTCCTGCACAAGTTTGACTTTAAAGAAGGACA
TGTCACATACCACAGAAGGTTCATCCGCACTGATGCTTACGTACGGGCAATGACTG
AGAAAAGGATCGTCATAACAGAATTTGGCACCTGTGCTTTCCCAGATCCCTGCAAG
AATATATTTTCCAGGTTTTTTTCTTACTTTCGAGGAGTAGAGGTTACTGACAATGCC
CTTGTTAATGTCTACCCAGTGGGGGAAGATTACTACGCTTGCACAGAGACCAACTTT
ATTACAAAGATTAATCCAGAGACCTTGGAGACAATTAAGCAGGTTGATCTTTGCAA
CTATGTCTCTGTCAATGGGGCCACTGCTCACCCCCACATTGAAAATGATGGAACCGT
TTACAATATTGGTAATTGCTTTGGAAAAAATTTTTCAATTGCCTACAACATTGTAAA
GATCCCACCACTGCAAGCAGACAAGGAAGATCCAATAAGCAAGTCAGAGATCGTT
GTACAATTCCCCTGCAGTGACCGATTCAAGCCATCTTACGTTCATAGTTTTGGTCTG
ACTCCCAACTATATCGTTTTTGTGGAGACACCAGTCAAAATTAACCTGTTCAAGTTC
CTTTCTTCATGGAGTCTTTGGGGAGCCAACTACATGGATTGTTTTGAGTCCAATGAA
ACCATGGGGGTTTGGCTTCATATTGCTGACAAAAAAAGGAAAAAGTACCTCAATAA
TAAATACAGAACTTCTCCTTTCAACCTCTTCCATCACATCAACACCTATGAAGACAA
TGGGTTTCTGATTGTGGATCTCTGCTGCTGGAAAGGATTTGAGTTTGTTTATAATTA
CTTATATTTAGCCAATTTACGTGAGAACTGGGAAGAGGTGAAAAAAAATGCCAGAA
AGGCTCCCCAACCTGAAGTTAGGAGATATGTACTTCCTTTGAATATTGACAAGGCTG
ACACAGGCAAGAATTTAGTCACGCTCCCCAATACAACTGCCACTGCAATTCTGTGC
AGTGACGAGACTATCTGGCTGGAGCCTGAAGTTCTCTTTTCAGGGCCTCGTCAAGCA
TTTGAGTTTCCTCAAATCAATTACCAGAAGTATTGTGGGAAACCTTACACATATGCG
TATGGACTTGGCTTGAATCACTTTGTTCCAGATAGGCTCTGTAAGCTGAATGTCAAA
ACTAAAGAAACTTGGGTTTGGCAAGAGCCTGATTCATACCCATCAGAACCCATCTTT
GTTTCTCACCCAGATGCCTTGGAAGAAGATGATGGTGTAGTTCTGAGTGTGGTGGTG
AGCCCAGGAGCAGGACAAAAGCCTGCTTATCTCCTGATTCTGAATGCCAAGGACTT
AAGTGAAGTTGCCCGGGCTGAAGTGGAGATTAACATCCCTGTCACCTTTCATGGAC
TGTTCAAAAAATCTTGA
SEQ ID NO: 2
ATGTCTATCCAGGTTGAGCATCCTGCTGGTGGTTACAAGAAACTGTTTGAAACTGTG
GAGGAACTGTCCTCGCCGCTCACAGCTCATGTAACAGGCAGGATCCCCCTCTGGCT
CACCGGCAGTCTCCTTCGATGTGGGCCAGGACTCTTTGAAGTTGGATCTGAGCCATT
TTACCACCTGTTTGATGGGCAAGCCCTCCTGCACAAGTTTGACTTTAAAGAAGGACA
CGTCACATACCACAGAAGGTTCATCCGCACTGATGCTTACGTACGGGCAATGACTG
AGAAAAGGATCGTCATAACAGAATTTGGCACCTGTGCTTTCCCAGATCCCTGCAAG
AATATATTTTCCAGGTTTTTTTCTTACTTTCGAGGAGTAGAGGTTACTGACAACGCC
CTTGTTAATGTCTACCCAGTGGGGGAAGATTACTACGCTTGCACAGAGACCAACTTT
ATTACAAAGATTAATCCAGAGACCTTGGAGACAATTAAGCAGGTTGATCTTTGCAA
CTATGTCTCTGTCAATGGGGCCACTGCTCACCCCCACATTGAAAATGATGGAACCGT
TTACAATATTGGTAATTGCTTTGGAAAAAATTTTTCAATTGCCTACAACATTGTAAA
GATCCCACCACTGCAAGCAGACAAGGAAGATCCAATAAGCAAGTCAGAGATCGTT
GTACAATTCCCCTGCAGTGACCGATTCAAGCCATCTTACGTTCATAGTTTTGGTCTG
ACTCCCAACTATATCGTTTTTGTGGAGACACCAGTCAAAATTAACCTGTTCAAGTTC
CTTTCTTCATGGAGTCTTTGGGGAGCCAACTACATGGATTGTTTTGAGTCCAATGAA
ACCATGGGGGTTTGGCTTCATATTGCTGACAAAAAAAGGAAAAAGTACCTCAATAA
TAAATACAGAACTTCTCCTTTCAACCTCTTCCATCACATCAACACCTATGAAGACAA
TGGGTTTCTGATTGTGGATCTCTGCTGCTGGAAAGGATTTGAGTTTGTTTATAATTA
CTTATATTTAGCCAATTTACGTGAGAACTGGGAAGAGGTGAAAAAAAATGCCAGAA
AGGCTCCCCAACCTGAAGTTAGGAGATATGTACTTCCTTTGAATATTGACAAGGCTG
ACACAGGCAAGAATTTAGTCACGCTCCCCAATACAACTGCCACTGCAATTCTGTGC
AGTGACGAGACTATCTGGCTGGAGCCTGAAGTTCTCTTTTCAGGGCCTCGTCAAGCA
TTTGAGTTTCCTCAAATCAATTACCAGAAGTATTGTGGGAAACCTTACACATATGCG
TATGGACTTGGCTTGAATCACTTTGTTCCAGATAGGCTCTGTAAGCTGAATGTCAAA
ACTAAAGAAACTTGGGTTTGGCAAGAGCCTGATTCATACCCATCAGAACCCATCTTT
GTTTCTCACCCAGATGCCTTGGAAGAAGATGATGGTGTAGTTCTGAGTGTGGTGGTG
AGCCCAGGAGCAGGACAAAAGCCTGCTTATCTCCTGATTCTGAATGCCAAGGACTT
AAGTGAAGTTGCCCGGGCTGAAGTGGAGATTAACATCCCTGTCACCTTTCACGGAC
TGTTCAAAAAATCTTGA
SEQ ID NO: 3
ATGTCCATCCAGGTGGAGCACCCAGCTGGAGGCTACAAGAAGCTGTTTGAAACTGT
GGAAGAACTGAGCAGCCCCCTGACAGCCCATGTGACAGGCAGAATCCCTCTGTGGC
TGACAGGCAGCCTGCTGAGATGTGGCCCAGGCCTGTTTGAGGTGGGCTCTGAGCCT
TTCTACCACCTGTTTGATGGCCAAGCCCTGCTCCACAAGTTTGATTTCAAGGAGGGC
CATGTGACCTACCACAGAAGATTCATCAGAACAGATGCCTATGTGAGGGCCATGAC
AGAGAAGAGGATAGTTATCACAGAGTTTGGCACCTGTGCCTTCCCTGACCCCTGCA
AGAACATCTTCAGCAGATTCTTCAGCTACTTCAGAGGAGTGGAAGTGACAGACAAT
GCCCTGGTCAATGTGTACCCTGTGGGAGAGGACTACTATGCCTGTACTGAGACCAA
CTTCATCACCAAGATCAACCCTGAAACCCTGGAAACCATCAAGCAGGTGGACCTGT
GCAACTATGTGTCAGTCAATGGAGCCACAGCCCACCCTCACATTGAGAATGATGGC
ACAGTTTACAACATAGGCAACTGCTTTGGCAAAAACTTCAGCATTGCCTACAACATT
GTGAAGATCCCCCCTCTGCAGGCTGACAAGGAGGACCCCATCAGCAAGTCTGAGAT
AGTGGTGCAGTTCCCATGCTCTGACAGATTCAAGCCCAGCTATGTGCACAGCTTTGG
CCTGACCCCAAACTACATTGTGTTTGTGGAAACCCCTGTGAAGATCAACCTGTTCAA
GTTCCTGAGCTCCTGGAGCCTGTGGGGAGCCAACTACATGGACTGCTTTGAAAGCA
ATGAGACCATGGGAGTGTGGCTGCACATTGCTGACAAGAAAAGAAAGAAGTACCT
GAACAACAAATACAGAACCAGCCCTTTCAACCTGTTCCACCACATCAACACCTATG
AGGACAATGGCTTCCTGATTGTGGACCTGTGCTGCTGGAAGGGCTTTGAGTTTGTGT
ACAACTACCTGTACCTGGCCAACCTGAGAGAAAACTGGGAGGAAGTGAAAAAAAA
TGCCAGAAAGGCCCCCCAGCCTGAGGTGAGGAGATATGTGCTGCCTCTGAACATAG
ACAAGGCTGACACAGGCAAGAACCTGGTGACCCTCCCCAACACCACAGCCACAGCC
ATCCTGTGCTCTGATGAGACCATCTGGCTGGAGCCTGAAGTGCTGTTCTCTGGCCCC
AGACAGGCCTTTGAGTTCCCTCAAATCAACTACCAGAAATACTGTGGCAAACCCTA
CACCTATGCCTATGGCCTGGGCCTGAACCACTTTGTCCCTGACAGACTGTGCAAGCT
GAATGTGAAAACCAAGGAGACCTGGGTCTGGCAGGAGCCTGACTCCTACCCTTCTG
AACCCATCTTTGTGAGCCACCCTGATGCCCTGGAGGAGGATGATGGAGTGGTGCTG
AGTGTGGTGGTCAGCCCTGGTGCTGGCCAGAAGCCTGCATACCTGCTAATCCTGAA
TGCCAAGGACCTGTCTGAAGTTGCCAGGGCTGAGGTGGAAATCAACATCCCTGTGA
CCTTCCATGGCCTTTTCAAGAAGAGCTGA
SEQ ID NO: 4
ATGAGCATCCAGGTGGAACATCCTGCTGGTGGCTACAAGAAACTGTTTGAGACAGT
GGAAGAACTGAGCAGCCCTCTGACAGCCCATGTGACAGGCAGAATCCCTCTGTGGC
TGACAGGCTCCCTGCTGAGATGTGGCCCTGGCCTGTTTGAAGTGGGCTCTGAGCCTT
TCTACCACCTGTTTGATGGACAGGCCCTGCTGCACAAGTTTGACTTCAAAGAGGGCC
ATGTGACCTACCACAGAAGATTCATCAGGACAGATGCCTATGTCAGAGCCATGACA
GAGAAGAGGATTGTGATCACTGAGTTTGGCACCTGTGCCTTTCCAGATCCTTGCAAG
AACATCTTCAGCAGATTCTTCAGCTACTTCAGAGGGGTTGAAGTGACAGACAATGC
CCTGGTCAATGTGTACCCTGTGGGAGAAGATTACTATGCCTGCACAGAGACAAACT
TCATCACCAAGATCAACCCTGAGACACTGGAAACCATCAAGCAGGTTGACCTGTGC
AACTATGTGTCTGTGAATGGGGCCACAGCTCACCCTCACATTGAGAATGATGGCAC
AGTGTACAACATTGGCAACTGCTTTGGCAAGAACTTCAGCATTGCCTACAACATAG
TGAAGATCCCACCTCTGCAGGCTGACAAAGAGGACCCCATCAGCAAGTCTGAGATT
GTGGTGCAGTTCCCCTGCTCTGACAGATTCAAGCCCAGCTATGTGCACAGCTTTGGC
CTGACACCTAACTACATTGTGTTTGTGGAAACCCCTGTGAAGATCAATCTGTTCAAG
TTCCTGAGCAGCTGGTCCCTGTGGGGAGCCAACTACATGGACTGCTTTGAGAGCAA
TGAGACAATGGGAGTGTGGCTGCACATTGCAGACAAGAAGAGAAAGAAGTACCTG
AACAACAAGTACAGGACAAGCCCCTTCAACCTGTTCCACCACATCAACACCTATGA
GGACAATGGCTTCCTGATTGTGGACCTGTGCTGCTGGAAGGGCTTTGAGTTTGTGTA
CAACTACCTGTACCTGGCCAACCTGAGGGAAAACTGGGAAGAAGTGAAGAAGAAT
GCCAGAAAGGCCCCTCAGCCTGAAGTTAGAAGATATGTGCTGCCCCTGAACATTGA
CAAGGCTGACACAGGCAAGAACCTGGTCACCCTGCCTAACACCACAGCCACAGCCA
TCCTGTGCTCTGATGAGACTATCTGGCTGGAACCTGAGGTGCTGTTCTCTGGCCCCA
GACAGGCCTTTGAGTTCCCTCAGATCAACTACCAGAAATACTGTGGCAAGCCCTAC
ACCTATGCCTATGGCCTGGGCCTGAACCACTTTGTGCCAGACAGACTGTGCAAGCT
GAATGTCAAGACCAAAGAGACATGGGTCTGGCAAGAGCCTGACAGCTACCCTTCTG
AGCCCATCTTTGTGTCTCACCCTGATGCTCTGGAAGAGGATGATGGGGTTGTGCTGT
CTGTGGTGGTGTCCCCTGGTGCTGGACAGAAGCCTGCCTATCTGCTGATCCTGAATG
CCAAGGACCTGTCTGAGGTGGCCAGAGCTGAGGTGGAAATCAACATCCCTGTGACC
TTCCATGGCCTGTTCAAGAAGTCCTGA
SEQ ID NO: 5
ATGAGCATCCAGGTGGAACATCCTGCTGGTGGCTACAAGAAACTGTTTGAGACAGT
GGAAGAACTGAGCAGCCCTCTGACAGCCCATGTGACAGGCAGAATCCCTCTGTGGC
TGACAGGCTCCCTGCTGAGATGTGGCCCTGGCCTGTTTGAAGTGGGCTCTGAGCCTT
TCTACCACCTGTTTGATGGACAGGCCCTGCTGCACAAGTTTGACTTCAAAGAGGGCC
ATGTGACCTACCACAGAAGATTCATCAGGACAGATGCCTATGTCAGAGCCATGACA
GAGAAGAGGATTGTGATCACTGAGTTTGGCACCTGTGCCTTTCCAGATCCTTGCAAG
AACATCTTCAGCAGATTCTTCAGCTACTTCAGAGGGGTTGAAGTGACAGACAATGC
CCTGGTCAATGTGTACCCTGTGGGAGAAGATTACTATGCCTGCACAGAGACAAACT
TCATCACCAAGATCAACCCTGAGACACTGGAAACCATCAAGCAGGTTGACCTGTGC
AACTATGTGTCTGTGAATGGGGCCACAGCTCACCCTCACATTGAGAATGATGGCAC
AGTGTACAACATTGGCAACTGCTTTGGCAAGAACTTCAGCATTGCCTACAACATAG
TGAAGATCCCACCTCTGCAGGCTGACAAAGAGGACCCCATCAGCAAGTCTGAGATT
GTGGTGCAGTTCCCCTGCTCTGACAGATTCAAGCCCAGCTATGTGCACAGCTTTGGC
CTGACACCTAACTACATTGTGTTTGTGGAAACCCCTGTGAAGATCAATCTGTTCAAG
TTCCTGAGCAGCTGGTCCCTGTGGGGAGCCAACTACATGGACTGCTTTGAGAGCAA
TGAGACAATGGGAGTGTGGCTGCACATTGCAGACAAGAAGAGAAAGAAGTACCTG
AACAACAAGTACCGGACAAGCCCCTTCAACCTGTTCCACCACATCAACACCTATGA
GGACAATGGCTTCCTGATTGTGGACCTGTGCTGCTGGAAGGGCTTTGAGTTCGTGTA
CAACTACCTGTACCTGGCCAACCTGAGGGAAAACTGGGAAGAAGTGAAGAAGAAT
GCCAGAAAGGCCCCTCAGCCTGAAGTTAGAAGATATGTGCTGCCCCTGAACATTGA
CAAGGCTGACACAGGCAAGAACCTGGTCACCCTGCCTAACACCACAGCCACAGCCA
TCCTGTGCTCTGATGAGACTATCTGGCTGGAACCTGAGGTGCTGTTCTCTGGCCCCA
GACAGGCCTTCGAGTTCCCTCAGATCAACTACCAGAAATACTGCGGCAAGCCCTAC
ACCTATGCCTATGGCCTGGGCCTGAACCACTTCGTGCCAGACAGACTGTGCAAGCT
GAATGTCAAGACCAAAGAGACATGGGTCTGGCAAGAGCCTGACAGCTACCCTTCTG
AGCCCATCTTTGTGTCTCACCCTGATGCTCTGGAAGAGGATGATGGGGTTGTGCTGT
CTGTGGTGGTGTCCCCTGGTGCTGGACAGAAGCCTGCCTATCTGCTGATCCTGAATG
CCAAGGACCTGTCTGAGGTGGCCAGAGCTGAGGTGGAAATCAACATCCCTGTGACC
TTCCATGGCCTGTTCAAGAAGTCCTGA
SEQ ID NO: 6
ATGTCCATCCAGGTGGAGCACCCAGCTGGAGGCTACAAGAAGCTGTTTGAGACTGT
GGAAGAACTGAGCAGCCCCCTGACAGCCCATGTGACAGGCAGGATCCCCCTGTGGC
TGACAGGCAGCCTGCTGAGATGTGGCCCAGGCCTGTTTGAGGTGGGCTCTGAGCCT
TTCTACCACCTGTTTGATGGCCAAGCCCTGCTCCACAAGTTCGATTTCAAAGAGGGC
CATGTGACCTACCACAGACGGTTCATCAGAACAGATGCCTATGTGAGAGCCATGAC
AGAGAAGAGAATTGTGATCACAGAATTTGGCACCTGTGCCTTCCCTGACCCCTGCA
AGAACATCTTCAGCAGATTCTTCAGCTACTTCAGAGGAGTGGAAGTGACAGACAAT
GCCCTGGTGAATGTGTACCCTGTGGGAGAAGACTACTATGCCTGTACTGAGACCAA
CTTCATCACCAAGATCAACCCTGAAACCCTGGAAACCATCAAGCAGGTGGACCTGT
GCAACTATGTGTCAGTCAATGGAGCCACAGCCCACCCTCACATCGAGAATGATGGC
ACAGTTTACAACATTGGCAACTGCTTTGGCAAAAACTTCAGCATCGCCTACAACATT
GTGAAGATCCCCCCTCTGCAGGCTGACAAAGAGGACCCCATCAGCAAGTCTGAGAT
AGTGGTGCAGTTCCCATGCTCTGACCGGTTCAAGCCCAGCTATGTGCACAGCTTTGG
CCTGACCCCAAACTACATTGTGTTTGTGGAAACCCCTGTCAAAATCAACCTGTTCAA
ATTCCTGAGCTCCTGGAGCCTGTGGGGAGCCAACTACATGGACTGCTTTGAAAGCA
ATGAGACCATGGGAGTGTGGCTGCACATTGCTGACAAGAAACGGAAGAAGTACCT
GAACAACAAGTACCGGACCAGCCCTTTCAACCTGTTCCACCACATCAACACCTATG
AGGACAATGGCTTCCTGATCGTGGACCTGTGCTGCTGGAAGGGCTTTGAGTTCGTGT
ACAACTACCTGTACCTGGCCAACCTGAGAGAAAACTGGGAGGAGGTGAAGAAGAA
TGCCAGAAAGGCCCCCCAGCCTGAAGTGAGGAGATATGTGCTGCCTCTGAACATAG
ACAAGGCTGACACAGGCAAGAACCTGGTGACCCTCCCTAACACCACAGCCACAGCC
ATCCTCTGCTCTGATGAGACCATCTGGCTGGAACCTGAAGTGCTGTTCTCTGGCCCC
AGACAGGCCTTTGAGTTCCCACAAATCAACTACCAGAAATACTGTGGCAAGCCCTA
CACCTACGCCTATGGCCTGGGCCTGAACCACTTTGTGCCTGACAGACTGTGCAAGCT
GAATGTGAAGACCAAGGAGACCTGGGTCTGGCAGGAGCCTGACTCCTACCCTTCTG
AACCCATCTTTGTCAGCCACCCTGATGCCCTGGAGGAGGATGATGGAGTGGTGCTG
AGTGTGGTGGTGAGCCCTGGTGCTGGCCAGAAGCCTGCATACCTGCTGATCCTGAA
TGCCAAGGACCTGTCTGAGGTTGCCAGAGCTGAGGTGGAAATCAACATCCCTGTCA
CCTTCCATGGCTTATTCAAGAAAAGCTGA
SEQ ID NO: 7
ATGAGCATCCAGGTTGAGCATCCTGCTGGTGGTTACAAGAAACTGTTTGAAACTGT
GGAGGAACTGTCCTCGCCGCTCACAGCTCATGTAACAGGCAGGATCCCCCTCTGGC
TCACCGGCAGTCTCCTTCGATGTGGGCCAGGACTCTTTGAAGTTGGATCTGAGCCAT
TTTACCACCTGTTTGATGGGCAAGCCCTCCTGCACAAGTTTGACTTTAAAGAAGGAC
ATGTCACATACCACAGAAGGTTCATCCGCACTGATGCTTACGTACGGGCAATGACT
GAGAAAAGGATCGTCATAACAGAATTTGGCACCTGTGCTTTCCCAGATCCCTGCAA
GAATATATTTTCCAGGTTTTTTTCTTACTTTCGAGGAGTAGAGGTTACTGACAACGC
CCTTGTTAATGTCTACCCAGTGGGGGAAGATTACTACGCTTGCACAGAGACCAACTT
TATTACAAAGATTAATCCAGAGACCTTGGAGACAATTAAGCAGGTTGATCTTTGCA
ACTATGTCTCTGTCAATGGGGCCACTGCTCACCCCCACATTGAAAATGATGGAACC
GTTTACAATATTGGTAATTGCTTTGGAAAAAATTTTTCAATTGCCTACAACATTGTA
AAGATCCCACCACTGCAAGCAGACAAGGAAGATCCAATAAGCAAGTCAGAGATCG
TTGTACAATTCCCCTGCAGTGACCGATTCAAGCCATCTTACGTTCATAGTTTTGGTCT
GACTCCCAACTATATCGTTTTTGTGGAGACACCAGTCAAAATTAACCTGTTCAAGTT
CCTTTCTTCATGGAGTCTTTGGGGAGCCAACTACATGGATTGTTTTGAGTCCAATGA
AACCATGGGGGTTTGGCTTCATATTGCTGACAAAAAAAGGAAAAAGTACCTCAATA
ATAAATACAGAACTTCTCCTTTCAACCTCTTCCATCACATCAACACCTATGAAGACA
ATGGGTTTCTGATTGTGGATCTCTGCTGCTGGAAAGGATTTGAGTTTGTTTATAATT
ACTTATATTTAGCCAATTTACGTGAGAACTGGGAAGAGGTGAAAAAAAATGCCAGA
AAGGCTCCCCAACCTGAAGTTAGGAGATATGTACTTCCTTTGAATATTGACAAGGCT
GACACAGGCAAGAATTTAGTCACGCTCCCCAATACAACTGCCACTGCAATTCTGTG
CAGTGACGAGACTATCTGGCTGGAGCCTGAAGTTCTCTTTTCAGGGCCTCGTCAAGC
ATTTGAGTTTCCTCAAATCAATTACCAGAAGTATTGTGGGAAACCTTACACATATGC
GTATGGACTTGGCTTGAATCACTTTGTTCCAGATAGGCTCTGTAAGCTGAATGTCAA
AACTAAAGAAACTTGGGTTTGGCAAGAGCCTGATTCATACCCATCAGAACCCATCT
TTGTTTCTCACCCAGATGCCTTGGAAGAAGATGATGGTGTAGTTCTGAGTGTGGTGG
TGAGCCCAGGAGCAGGACAAAAGCCTGCTTATCTCCTGATTCTGAATGCCAAGGAC
TTAAGTGAAGTTGCCCGGGCTGAAGTGGAGATTAACATCCCTGTCACCTTTCATGGA
CTGTTCAAAAAATCTTAATAA
SEQ ID NO: 8
ATGTCTATTCAAGTCGAGCACCCAGCGGGGGGATATAAAAAGCTTTTCGAAACGGT
GGAGGAGCTGAGCTCCCCCCTTACGGCGCATGTTACGGGGCGCATACCTCTGTGGC
TCACGGGATCATTGCTTCGCTGCGGACCCGGATTGTTCGAGGTTGGCAGTGAACCAT
TCTACCATCTCTTCGATGGTCAGGCATTGCTTCATAAATTTGATTTCAAAGAAGGAC
ACGTCACATATCATCGCAGGTTCATCCGGACAGATGCGTACGTTCGCGCCATGACA
GAAAAGCGCATTGTAATAACTGAGTTTGGGACATGTGCATTTCCTGACCCTTGTAAG
AATATATTCAGCCGCTTTTTCAGCTATTTTAGAGGCGTTGAGGTTACTGACAATGCG
CTCGTGAACGTCTATCCAGTAGGTGAAGATTATTACGCCTGTACTGAGACTAATTTC
ATCACTAAAATTAATCCAGAAACTTTGGAGACCATAAAGCAGGTTGATCTCTGTAA
CTATGTCTCAGTGAATGGCGCTACAGCGCACCCGCACATAGAAAACGATGGAACGG
TTTACAATATAGGCAACTGTTTTGGTAAGAATTTTAGCATTGCTTACAACATAGTCA
AGATACCTCCTCTCCAAGCCGATAAAGAGGACCCGATATCCAAATCAGAGATTGTT
GTCCAATTCCCGTGCAGTGATAGATTCAAACCGTCTTACGTCCACAGTTTTGGCCTG
ACACCCAATTACATTGTTTTTGTTGAAACACCTGTTAAGATAAATCTCTTTAAATTTC
TGTCTTCTTGGAGTCTGTGGGGGGCAAATTACATGGATTGTTTCGAGTCTAACGAGA
CGATGGGAGTCTGGCTTCATATAGCAGATAAAAAGCGCAAAAAGTATTTGAATAAC
AAGTACCGGACGAGCCCGTTCAATTTGTTCCACCATATCAACACTTACGAGGATAA
CGGGTTTCTGATCGTCGACCTTTGCTGTTGGAAAGGGTTCGAGTTCGTGTATAACTA
CCTCTACTTGGCGAACCTTCGGGAAAATTGGGAGGAAGTTAAGAAGAACGCAAGA
AAGGCCCCGCAGCCAGAAGTCCGAAGGTATGTTCTGCCGTTGAATATCGACAAAGC
CGACACTGGAAAGAACCTCGTTACGCTTCCCAATACCACGGCTACCGCGATCTTGT
GCAGTGACGAAACAATTTGGCTGGAGCCCGAGGTGTTGTTTTCTGGCCCAAGGCAA
GCCTTTGAATTCCCACAGATAAATTATCAAAAATATTGTGGAAAGCCCTACACCTAC
GCTTATGGACTCGGTCTCAACCATTTTGTTCCAGATCGACTTTGCAAGCTGAATGTA
AAGACCAAAGAAACCTGGGTTTGGCAAGAACCCGATTCCTACCCCAGTGAACCGAT
CTTTGTTTCCCATCCCGACGCCCTCGAAGAAGACGACGGAGTIGTCTTGTCCGTTGT
GGTGAGCCCCGGTGCAGGACAGAAGCCCGCTTATCTTTTGATTCTTAATGCCAAAG
ATTTGTCAGAAGTAGCGCGGGCCGAGGTAGAGATCAACATACCTGTTACTTTCCAT
GGGTTGTTCAAAAAGAGTTGA
SEQ ID NO: 9
ATGAGCATCCAGGTGGAACATCCTGCCGGCGGATACAAGAAACTGTTCGAGACAGT
GGAAGAACTGAGCAGCCCTCTGACAGCCCACGTGACAGGCAGAATCCCTCTGTGGC
TGACCGGCAGCCTGCTGAGATGTGGACCTGGCCTGTTTGAAGTGGGCAGCGAGCCT
TTCTACCACCTGTTCGATGGACAGGCCCTGCTGCACAAGTTCGACTTCAAAGAGGG
CCACGTCACCTACCACCGGCGGTTCATTAGAACCGATGCCTACGTGCGGGCCATGA
CCGAGAAGAGAATCGTGATCACCGAGTTCGGCACCTGTGCCTTTCCAGATCCTTGC
AAGAACATCTTCAGCCGGTTCTTCAGCTACTTCAGAGGCGTGGAAGTGACCGACAA
CGCCCTGGTCAATGTGTACCCCGTGGGCGAAGATTACTACGCCTGCACCGAGACAA
ACTTCATCACCAAGATCAACCCCGAGACACTGGAAACCATCAAGCAGGTTGACCTG
TGCAACTACGTGTCCGTGAACGGCGCCACAGCTCACCCTCACATCGAGAATGATGG
CACCGTGTACAACATCGGCAACTGCTTCGGCAAGAACTTCTCTATCGCCTACAATAT
CGTGAAGATCCCACCTCTGCAGGCCGACAAAGAGGACCCCATCAGCAAGAGCGAG
ATCGTGGTGCAGTTCCCCTGCAGCGACAGATTCAAGCCCAGCTACGTGCACAGCTT
CGGCCTGACACCTAACTACATCGTGTTCGTGGAAACCCCTGTGAAGATCAATCTGTT
CAAGTTCCTGAGCAGCTGGTCCCTGTGGGGCGCCAACTACATGGATTGCTTCGAGA
GCAACGAGACAATGGGCGTGTGGCTGCACATTGCCGACAAGAAGCGGAAGAAGTA
CCTGAACAACAAGTACCGGACAAGCCCCTTCAACCTGTTCCACCACATCAACACCT
ACGAGGACAACGGCTTCCTGATCGTGGACCTGTGTTGCTGGAAGGGCTTCGAGTTC
GTGTACAATTACCTGTACCTGGCCAACCTGCGCGAGAACTGGGAAGAAGTGAAGAA
GAACGCCCGGAAGGCCCCTCAGCCTGAAGTGCGAAGATATGTGCTGCCCCTGAACA
TCGACAAGGCCGACACCGGCAAGAATCTGGTCACCCTGCCTAATACCACCGCCACC
GCCATCCTGTGTAGCGACGAAACCATCTGGCTGGAACCCGAGGTGCTGTTCTCTGG
ACCTAGACAGGCCTTCGAGTTTCCCCAGATCAACTACCAGAAGTACTGCGGCAAGC
CCTACACCTACGCCTATGGCCTGGGCCTGAATCACTTCGTGCCCGACAGACTGTGCA
AGCTGAACGTCAAGACCAAAGAGACATGGGTCTGGCAAGAGCCCGACAGCTACCC
TAGCGAGCCCATCTTTGTGTCTCACCCCGACGCTCTGGAAGAGGACGATGGCGTTGT
GCTGAGCGTGGTGGTTTCTCCTGGCGCCGGACAGAAACCTGCCTACCTGCTGATCCT
GAACGCCAAGGACCTGAGCGAAGTGGCCAGAGCCGAGGTGGAAATCAACATCCCC
GTGACCTTCCACGGCCTGTTCAAGAAGTCCTAATAA
SEQ ID NO: 10
ATGAGCATCCAGGTGGAGCACCCCGCCGGCGGCTACAAGAAGCTGTTCGAGACCGT
GGAGGAGCTGAGCAGCCCCCTGACCGCCCACGTGACCGGCCGCATCCCCCTGTGGC
TGACCGGCAGCCTGCTGCGCTGCGGCCCCGGCCTGTTCGAGGTGGGCAGCGAGCCC
TTCTACCACCTGTTCGACGGCCAGGCCCTGCTGCACAAGTTCGACTTCAAGGAGGG
CCACGTGACCTACCACCGCCGCTTCATCCGCACCGACGCCTACGTGCGCGCCATGA
CCGAGAAGCGCATCGTGATCACCGAGTTCGGCACCTGCGCCTTCCCCGACCCCTGC
AAGAACATCTTCAGCCGCTTCTTCAGCTACTTCCGCGGCGTGGAGGTGACCGACAA
CGCCCTGGTGAACGTGTACCCCGTGGGCGAGGACTACTACGCCTGCACCGAGACCA
ACTTCATCACCAAGATCAACCCCGAGACCCTGGAGACCATCAAGCAGGTGGACCTG
TGCAACTACGTGAGCGTGAACGGCGCCACCGCCCACCCCCACATCGAGAACGACGG
CACCGTGTACAACATCGGCAACTGCTTCGGCAAGAACTTCAGCATCGCCTACAACA
TCGTGAAGATCCCCCCCCTGCAGGCCGACAAGGAGGACCCCATCAGCAAGAGCGA
GATCGTGGTGCAGTTCCCCTGCAGCGACCGCTTCAAGCCCAGCTACGTGCACAGCTT
CGGCCTGACCCCCAACTACATCGTGTTCGTGGAGACCCCCGTGAAGATCAACCTGTT
CAAGTTCCTGAGCAGCTGGAGCCTGTGGGGCGCCAACTACATGGACTGCTTCGAGA
GCAACGAGACCATGGGCGTGTGGCTGCACATCGCCGACAAGAAGCGCAAGAAGTA
CCTGAACAACAAGTACCGCACCAGCCCCTTCAACCTGTTCCACCACATCAACACCT
ACGAGGACAACGGCTTCCTGATCGTGGACCTGTGCTGCTGGAAGGGCTTCGAGTTC
GTGTACAACTACCTGTACCTGGCCAACCTGCGCGAGAACTGGGAGGAGGTGAAGAA
GAACGCCCGCAAGGCCCCCCAGCCCGAGGTGCGCCGCTACGTGCTGCCCCTGAACA
TCGACAAGGCCGACACCGGCAAGAACCTGGTGACCCTGCCCAACACCACCGCCACC
GCCATCCTGTGCAGCGACGAGACCATCTGGCTGGAGCCCGAGGTGCTGTTCAGCGG
CCCCCGCCAGGCCTTCGAGTTCCCCCAGATCAACTACCAGAAGTACTGCGGCAAGC
CCTACACCTACGCCTACGGCCTGGGCCTGAACCACTTCGTGCCCGACCGCCTGTGCA
AGCTGAACGTGAAGACCAAGGAGACCTGGGTGTGGCAGGAGCCCGACAGCTACCC
CAGCGAGCCCATCTTCGTGAGCCACCCCGACGCCCTGGAGGAGGACGACGGCGTGG
TGCTGAGCGTGGTGGTGAGCCCCGGCGCCGGCCAGAAGCCCGCCTACCTGCTGATC
CTGAACGCCAAGGACCTGAGCGAGGTGGCCCGCGCCGAGGTGGAGATCAACATCC
CCGTGACCTTCCACGGCCTGTTCAAGAAGAGCTAA
SEQ ID NO: 11
MSIQVEHPAGGYKKLFETVEELSSPLTAHVTGRIPLWLTGSLLRCGPGLFEVGSEPFYHL
FDGQALLHKFDFKEGHVTYHRRFIRTDAYVRAMTEKRIVITEFGTCAFPDPCKNIFSRFF
SYFRGVEVTDNALVNVYPVGEDYYACTETNFITKINPETLETIKQVDLCNYVSVNGATA
HPHIENDGTVYNIGNCFGKNFSIAYNIVKIPPLQADKEDPISKSEIVVQFPCSDRFKPSYV
HSFGLTPNYIVFVETPVKINLFKFLSSWSLWGANYMDCFESNETMGVWLHIADKKRKK
YLNNKYRTSPFNLFHHINTYEDNGFLIVDLCCWKGFEFVYNYLYLANLRENWEEVKKN
ARKAPQPEVRRYVLPLNIDKADTGKNLVTLPNTTATAILCSDETIWLEPEVLFSGPRQAF
EFPQINYQKYCGKPYTYAYGLGLNHFVPDRLCKLNVKTKETWVWQEPDSYPSEPIFVS
HPDALEEDDGVVLSVVVSPGAGQKPAYLLILNAKDLSEVARAEVEINIPVTFHGLFKKS
SEQ ID NO: 12
CGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATT
GACGTCAATGGGTGGACTATTTACGGTAAACTGCCCACTTGGCAGTACATCAAGTG
TATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGTCGAGGTGAGCCCCACGTTCTGCTTCACTCTCCCC
ATCTCCCCCCCCTCCCCACCCCCAATTTTGTATTTATTTATTTTTTAATTATTTTGTGC
AGCGATTGGGGGCGGGGGGGGGGGGGGGGCGCGCGCCAGGCGGGGCGGGGCGGG
GCGAGGGGCGGGGCGGGGCGAGGCGGAGAGGTGCGGCGGCAGCCAATCAGAGCG
GCGCGCTCCGAAAGTTTCCTTTTATGGCGAGGCGGCGGCGGCGGCGGCCCTATAAA
AAGCGAAGCGCGCGGCGGGCGGGAGTCGCTGCGACGCTGCCTTCGCCCCGTGCCCC
GCTCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGTTTAAT
GACGGCTTGTTTCTTTTCTGTGGCTGCGTGAAAGCCTTGAGGGGCTCCGGGAGGGCC
CTTTGTGCGGGGGGGAGCGGCTCGGGGGGTGCGTGCGTGTGTGTGTGCGTGGGGAG
CGCCGCGTGCGGCCCGCGCTGCCCGGCGGCTGTGAGCGCTGCGGGCGCGGCGCGGG
GCTTTGTGCGCTCCGCAGTGTGCGCGAGGGGAGCGCGGCCGGGGGCGGTGCCCCGC
GGTGCGGGGGGGGCTGCGAGGGGAACAAAGGCTGCGTGCGGGGTGTGTGCGTGGG
GGGGTGAGCAGGGGGTGTGGGCGCGGCGGTCGGGCTGTAACCCCCCCCTGCACCCC
CCTCCCCGAGTTGCTGAGCACGGCCCGGCTTCGGGTGCGGGGCTCCGTACGGGGCG
TGGCGCGGGGCTCGCCGTGCCGGGCGGGGGGTGGCGGCAGGTGGGGGTGCCGGGC
GGGGCGGGGCCGCCTCGGGCCGGGGAGGGCTCGGGGGAGGGGCGCGGCGGCCCCC
GGAGCGCCGGCGGCTGTCGAGGCGCGGCGAGCCGCAGCCAATTGCCTTTTATGGTA
ATCGTGCGAGAGGGCGCAGGGACTTCCTTTGTCCCAAATCTGTGCGGAGCCGAAAT
CTGGGAGGCGCCGCCGCACCCCCTCTAGCGGGCGCGGGGCGAAGCGGTGCGGCGC
CGGCAGGAAGGAAATCGGGCGGGGAGGGCCTTCGTGCGTCGCCGCGCCGCCGTCCC
CTTCTCCCTCTCCAGCCTCGGGGCTGTCCGCGGGGGGACGGCTGCCTTCGGGGGGG
ACGGGGCAGGGCGGGGTTCGGCTTCTGGCGTGTGACCGGCGGCTCTAGAGCCTCTG
CTAACCATGTTCATGCCTTCTTCTTTTTCCTACAG
SEQ ID NO: 13
GGGTGGCATCCCTGTGACCCCTCCCCAGTGCCTCTCCTGGCCCTGGAAGTTGCCACT
CCAGTGCCCACCAGCCTTGTCCTAATAAAATTAAGTTGCAATCCATTTTGTCTGACT
AGGTGTCCTTCTATAATATTATGGGGTGGAGGGGGGTGGTATGGAGCAAGGGGCAA
GTTGGGAAGACAACCTGTAGGGCCTGCGGGGTCTATTGGGAACCAAGCTGGAGTGC
AGTGGCACAATCTTGGCTCACTGCAATCTCCGCCTCCTGGGTTCAAGCGATTCTCCT
GCCTCAGCCTCCCGAGTTGTTGGGATTCCAGGCATGCATGACCAGGCTCAGCTAATT
TTTGTTTTTTTGGTAGAGACGGGGTTTCACCATATTGGCCAGGCTGGTCTCCAACTC
CTAATCTCAGGTGATCTACCCACCTTGGCCTCCCAAATTGCTGGGATTACAGGCGTG
AACCACTGCTCCCTTCCCTGTCCTT
SEQ ID NO: 14
CTGTGCCTTCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGG
SEQ ID NO: 15
AACTTGTTTATTGCAGCTTATAATGGTTACAAATAAAGCAATAGCATCACAAATTTC
ACAAATAAAGCATTTTTTTCACTGCATTCTAGTTGTGGTTTGTCCAAACTCATCAAT
GTATCTTATCATGTCTGGATC
SEQ ID NO: 16
AATCAACCTCTGGATTACAAAATTTGTGAAAGATTGACTGGTATTCTTAACTATGTT
GCTCCTTTTACGCTATGTGGATACGCTGCTTTAATGCCTTTGTATCATGCTATTGCTT
CCCGTATGGCTTTCATTTTCTCCTCCTTGTATAAATCCTGGTTGCTGTCTCTTTATGA
GGAGTTGTGGCCCGTTGTCAGGCAACGTGGCGTGGTGTGCACTGTGTTTGCTGACGC
AACCCCCACTGGTTGGGGCATTGCCACCACCTGTCAGCTCCTTTCCGGGACTTTCGC
TTTCCCCCTCCCTATTGCCACGGCGGAACTCATCGCCGCCTGCCTTGCCCGCTGCTG
GACAGGGGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGGACGTCCT
TCTGCTACGTCCCTTCGGCCCTCAATCCAGCGGACCTTCCTTCCCGCGGCCTGCTGC
CGGCTCTGCGGCCTCTTCCGCGTCTTCGCCTTCGCCCTCAGACGAGTCGGATCTCCC
TTTGGGCCGCCTCCCCGC
SEQ ID NO: 17
MAADGYLPDWLEDTLSEGIRQWWKLKPGPPPPKPAERHKDDSRGLVLPGYKYLGPFN
GLDKGEPVNEADAAALEHDKAYDRQLDSGDNPYLKYNHADAEFQERLKEDTSFGGNL
GRAVFQAKKRVLEPLGLVEEPVKTAPGKKRPVEHSPVEPDSSSGTGKAGQQPARKRLN
FGQTGDADSVPDPQPLGQPPAAPSGLGTNTMATGSGAPMADNNEGADGVGNSSGNW
HCDSTWMGDRVITTSTRTWALPTYNNHLYKQISSQSGASNDNHYFGYSTPWGYFDFNR
FHCHFSPRDWQRLINNNWGFRPKRLNFKLFNIQVKEVTQNDGTTTIANNLTSTVQVFTD
SEYQLPYVLGSAHQGCLPPFPADVFMVPQYGYLTLNNGSQAVGRSSFYCLEYFPSQML
RTGNNFTFSYTFEDVPFHSSYAHSQSLDRLMNPLIDQYLYYLSRTNTPSGTTTQSRLQFS
QAGASDIRDQSRNWLPGPCYRQQRVSKTSADNNNSEYSWTGATKYHLNGRDSLVNPG
PAMASHKDDEEKFFPQSGVLIFGKQGSEKTNVDIEKVMITDEEEIRTTNPVATEQYGSVS
TNLQRGNRQAATADVNTQGVLPGMVWQDRDVYLQGPIWAKIPHTDGHFHPSPLMGG
FGLKHPPPQILIKNTPVPANPSTTFSAAKFASFITQYSTGQVSVEIEWELQKENSKRWNPE
IQYTSNYNKSVNVDFTVDTNGVYSEPRPIGTRYLTRNL
SEQ ID NO: 18
TAPGKKRPVEHSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTRTWALPTY
NNHLYKQISSQSGASNDNHYFGYSTPWGYFDFNRFHCHFSPRDWQRLINNNWGFRPKR
LNFKLFNIQVKEVTQNDGTTTIANNLTSTVQVFTDSEYQLPYVLGSAHQGCLPPFPADV
FMVPQYGYLTLNNGSQAVGRSSFYCLEYFPSQMLRTGNNFTFSYTFEDVPFHSSYAHSQ
SLDRLMNPLIDQYLYYLSRTNTPSGTTTQSRLQFSQAGASDIRDQSRNWLPGPCYRQQR
VSKTSADNNNSEYSWTGATKYHLNGRDSLVNPGPAMASHKDDEEKFFPQSGVLIFGKQ
GSEKTNVDIEKVMITDEEEIRTTNPVATEQYGSVSTNLQRGNRQAATADVNTQGVLPG
MVWQDRDVYLQGPIWAKIPHTDGHFHPSPLMGGFGLKHPPPQILIKNTPVPANPSTTFS
AAKFASFITQYSTGQVSVEIEWELQKENSKRWNPEIQYTSNYNKSVNVDFTVDTNGVY
SEPRPIGTRYLTRNL
SEQ ID NO: 19
MATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKL
FNIQVKEVTQNDGTTTIANNLTSTVQVFTDSEYQLPYVLGSAHQGCLPPFPADVFMVPQ
YGYLTLNNGSQAVGRSSFYCLEYFPSQMLRTGNNFTFSYTFEDVPFHSSYAHSQSLDRL
MNPLIDQYLYYLSRTNTPSGTTTQSRLQFSQAGASDIRDQSRNWLPGPCYRQQRVSKTS
ADNNNSEYSWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGVLPGMVWQ
DRDVYLQGPIWAKIPHTDGHFHPSPLMGGFGLKHPPPQILIKNTPVPANPSTTFSAAKFA
SFITQYSTGQVSVEIEWELQKENSKRWNPEIQYTSNYNKSVNVDFTVDTNGVYSEPRPI
GTRYLTRNL

1-72. (canceled)

73. A recombinant adeno-associated virus (rAAV) particle, comprising a polynucleotide sequence that comprises a coding sequence of RPE65 polypeptide, wherein the coding sequence is codon-optimized and contains an altered number of CpG dinucleotides as compared to a wildtype RPE65 nucleotide sequence, and wherein the coding sequence has at least 95% sequence identity to a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

74. The rAAV particle of claim 73, wherein the coding sequence is selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

75. The rAAV particle of claim 73, wherein the coding sequence comprises less than 20 CpG dinucleotides.

76. The rAAV particle of claim 73, wherein the polynucleotide sequence further comprises a promoter, where the promoter is operably linked to the coding sequence, and wherein the promoter is CMV, CAG, MNDU3, PGK, EF1a, Ubc promoter, or an ocular tissue specific promoter.

77. The rAAV particle of claim 76, wherein the ocular tissue specific promoter is selected from RPE65 gene promoter, human retinal binding protein (CRALBP) gene promoter, murine 11-cis-retinol dehydrogenase (RDH) gene promoter, rhodopsin promoter, rhodopsin kinase promoter, tissue inhibitor of metalloproteinase 3 (Timp3) promoter, photoreceptor retinol binding protein promoter, vitelliform macular dystrophy 2 promoter, or interphotoreceptor retinoid-binding protein (IRBP) promoter.

78. The rAAV particle of claim 73, wherein the polynucleotide sequence further comprises a WPRE sequence at the 3′ end, or a stuffer sequence, or a poly(A) sequence at the 3′ end selected from the group consisting of SV40pA, hGHpA, and bGHpA, and wherein the polynucleotide sequence comprises not more than 300 CpG dinucleotides.

79. A composition comprising:
(i) a first polynucleotide encoding an adeno-associated virus (AAV) protein, and
(ii) a second polynucleotide comprising a sequence encoding a RPE65 polypeptide, wherein the sequence encoding the RPE65 polypeptide is codon-optimized and contains an altered number of CpG dinucleotides as compared to a wildtype RPE65 nucleotide sequence, wherein the sequence encoding the RPE65 polypeptide has at least 95% sequence identity to a sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

80. The composition of claim 79, wherein the sequence encoding the RPE65 polypeptide is selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

81. The composition of claim 79, wherein the sequence encoding the RPE65 polypeptide comprises less than 20 CpG dinucleotides.

82. The composition of claim 79, wherein the AAV protein is a serotype 2 AAV protein, a serotype 5 AAV protein, a serotype 8 AAV protein, or fragments or variants thereof.

83. The composition of claim 79, wherein the first polynucleotide is codon-optimized.

84. The composition of claim 79, wherein the second polynucleotide comprises a promoter, wherein the promoter is operably linked to the sequence encoding the RPE65 polypeptide, and wherein the promoter is CMV, CAG, MNDU3, PGK, EF1a, Ubc promoter, or an ocular tissue specific promoter.

85. The composition of claim 84, wherein the ocular tissue specific promoter is selected from RPE65 gene promoter, human retinal binding protein (CRALBP) gene promoter, murine 11-cis-retinol dehydrogenase (RDH) gene promoter, rhodopsin promoter, rhodopsin kinase promoter, tissue inhibitor of metalloproteinase 3 (Timp3) promoter, photoreceptor retinol binding protein promoter, vitelliform macular dystrophy 2 promoter, or interphotoreceptor retinoid-binding protein (IRBP) promoter.

86. The composition of claim 79, wherein the second polynucleotide further comprises a WPRE sequence at the 3′ end, or a stuffer sequence, or a poly(A) sequence at the 3′ end selected from the group consisting of SV40pA, hGHpA, and bGHpA, and wherein the second polynucleotide comprises less than 300 CpG dinucleotides.

87. A host cell comprising an rAAV particle of claim 73.

88. A host cell comprising a composition of claim 79.

89. A method for preparing an rAAV particle, comprising introducing the composition of claim 79 in a host cell, wherein the host cell is Sf9 cell, HEK293 cell, or a cell derived from HEK293.

90. A pharmaceutical composition for treating Leber congenital amaurosis (LCA), comprising an rAAV particle of claim 73, and a pharmaceutically acceptable carrier.

91. A method for treating Leber congenital amaurosis (LCA) in a subject in need thereof, comprising administering a therapeutically effective amount of the pharmaceutical composition of claim 90 to the subject.

92. The method of claim 91, wherein the therapeutically effective amount is 1×10⁹ to 1×10¹³ of vector genomes (vg) for each eye.
